(19) United States
(12) Patent Application Publication
(10) Pub. No.: US 2022/0354818 A1
(43) Pub. Date: Nov. 10, 2022

(54) METHODS FOR TREATING BRAIN INJURY OR COGNITIVE DYSFUNCTION BY PHARMACOLOGICAL ENHANCMENT OF M-TYPE POTASSIUM ION CURRENTS IN NEURONS

(71) Applicant: Advanced Neuroresearch Therapeutics LLC, Austin, TX (US)

(72) Inventors: Mark Shapiro, San Antonio, TX (US); Fabio A. Vigil, San Antonio, TX (US)

(21) Appl. No.: 17/734,777

(22) Filed: May 2, 2022

Related U.S. Application Data
(63) Continuation-in-part of application No. 16/799,787, filed on Feb. 24, 2020, now abandoned.
Application No. 16/799,787 is a continuation-in-part of application No. 16/749,960, filed on Jan. 22, 2020, now abandoned.
(60) Provisional application No. 62/822,752, filed on Mar. 22, 2019.

(51) Int. Cl. A61K 31/27; A61P 25/00; A61K 45/06

(52) U.S. Cl. CPC A61K 31/27; A61P 25/00; A61K 45/06

(57) ABSTRACT

A method for the prevention of brain damage and cognitive dysfunction after blunt or blast types of TBI by a single systemic dose application either intravenous (i.v.) or intraperitoneal (i.p.) of a pharmacological “opener” of KCNQ (“M-type”) potassium ion channels in brain.

### PRIORITY

The present application is related to, claims the priority benefit of, and is a continuation-in-part application of U.S. patent application Ser. No. 16/799,787 filed Feb. 24, 2020, which is related to, a continuation-in-part application of, and claims the priority benefit of U.S. patent application Ser. No. 16/749,960 filed Jan. 22, 2020, which claims the priority benefit of U.S. Provisional Application No. 62/822,752, filed Mar. 22, 2019. The contents of each of the aforementioned priority applications are hereby expressly incorporated herein by reference in in their entireties.

### STATEMENT REGARDING FEDERALLY SPONSORED RESEARCH OR DEVELOPMENT

This invention was made with Government support under Grant No. W81WH-15-1-0284 awarded by the U.S. Department of Defense. The Government has certain rights in this invention.

### TECHNICAL FIELD

The present disclosure relates to traumatic brain injury (TBI) and methods for the prevention of brain damage and dysfunction after blunt or blast types of TBI by a administration of a pharmacological “opener” of KCNQ (Kv7, “M-type”) potassium ion channels (“M-channels”) in a brain.

### BACKGROUND

Nearly 3 million people in the U.S.A. suffer a traumatic brain injury (TBI) annually. However, there are no pre- or post-TBI treatment options available to prevent, protect against, delay or reduce brain damage after a TBI, leading to widespread neurological disease, such as seizures/epilepsy, and psychiatric disorders that include destruction of family units and in some tragic cases, suicides.

Besides acute mortality, TBIs can result in post-traumatic seizures (PTS) as well as post-concussion syndrome and the impairment in cognitive domains such as memory, processing speed, affect, impulse control, prediction/planning and other executive functions as measured by traditional neuropsychological instruments. Compounding the problem, PTS can accelerate the process of epileptogenesis by exacerbating cellular metabolic crisis, which can further increase brain damage.

TBI-induced tissue damage and cellular death can also lead to inflammatory/immunological responses in the brain and breakdown of the blood-brain barrier (BBB), promoting secondary brain damage. Among the major components of the inflammatory response are microglial over-activation (microgliosis), hyper-expression of the CD40 receptor on antigen-presenting cells of the immune system and macrophage infiltration, representing the first lines of defense after tissue damage. Further, TBI is known to induce acute hypoperfusion, due to vascular damage, which can lead to further cellular metabolic stress.

The physiological effects resulting from TBI can be detected immediately in some cases, but can also develop slowly over minutes, hours, weeks, months or even years post-injury. In some cases, the development of epilepsy following TBIs has been observed to have a latent period up to 10 years. Pathological processes can evolve slowly, making them difficult to detect in stages when interventions could be most effective.

A treatment is clearly needed to prevent these pathological outcomes, ideally one that can be administered within hours after a TBI is sustained, does not result in harmful side effects, and is capable of preventing or decreasing TBI-induced brain damage.

### BRIEF SUMMARY

The present disclosure provides a method for the prevention of brain damage and dysfunction after blunt or blast types of traumatic brain injury (TBI) by a single systemic dose application (i.v. or i.p.) of a pharmacological “opener” of KCNQ (Kv7, “M-type”) potassium ion channels (“M-channels”) in brain. Such brain damage and dysfunction can include post-traumatic seizures, a maladaptive inflammatory response, microgliosis, astrogliosis, widespread neuronal death, breakdown of the blood-brain-barrier, post-traumatic epilepsy, cognitive and locomotor dysfunction, mood changes, early Alzheimer's Disease and loss of quality of life, including suicides. Aspects of the present disclosure was included in a publication Vigil et al., Prevention of Brain Damage After Traumatic Brain Injury by Pharmacological Enhancement of KCNQ (Kv7, “M-type”) K⁺ Currents in Neurons, J Cereb Blood Flow Metab 40(6): 1256-1273, Jul. 4, 2019, which is incorporated herein by reference.

The present disclosure does not concern one particular opener molecule, but rather to the approach that is most likely applicable to many of them. Such opener molecules include retigabine (RTG), (2-amino-4-(4-fluorobenzylamino)-1-ethoxycarbonylaminobenzene, an aminoalkyl thiazole derivative (known as Ezogabine in Europe and sold in the USA under the trade name, Potiga, formerly FDA approved but now off the market as an anti-convulsant), ICA-069673 ([N-(2-chloro-5-pyrimidinyl)-3,4-difluorobenzamide]) and ICA-27243, and its derivatives, (2-amino-4-(arylamino-phenyl) carbamates (SF0034, RL648_81), and its derivatives, and other such “openers” (agonists) of KCNQ2-5 potassium ion channels that increase their currents in all known neurons of the central and peripheral nervous systems.

Seizures are very common after a TBI, making further seizures and development of epilepsy disease more likely. The present disclosure demonstrates that TBI-induced hyperexcitability and ischemia/hypoxia lead to cellular metabolic stress, cell death and a maladaptive inflammatory response that causes further downstream morbidity, and development of long-term epilepsy. Using a mouse controlled closed-cortical impact blunt TBI model, and a “blast-tube” TBI model, systemic administration of the prototype M-channel “opener,” retigabine (RTG), 30 minutes after TBI, reduces the post-TBI cascade of events including spontaneous seizures, enhanced susceptibility to further seizures, cellular metabolic stress, harmful immunological/inflammatory responses, blood-brain barrier (BBB) breakdown, neuronal death, neuronal necrosis/apoptosis and development of long-term epilepsy disease.

There are currently no treatments to prevent brain damage after a TBI, leading to widespread neurological disease, such as seizures/epilepsy, cognitive dysfunction, psychiatric disorders such as depression, and suicides. In certain embodiments, a method is provided for treating brain injury or dysfunction (e.g., cognitive or locomotive) resulting from at least one TBI comprising administering a therapeutically effective amount of a compound comprising an M-channel opener to a subject after the subject experiences a TBI. The M-channel is a compound that upregulates at lest a KCNQ2 subunit, a KCNQ3 subunit, or both of an M-channel of a brain of the subject.

In certain embodiments, the at least one TBI is selected from the group consisting of: blast injury, blunt trauma, Shaken Baby syndrome, concussion, and concussion syndrome.

The present disclosure is the first treatment that can prevent these pathological outcomes, and can be applied minutes or hours after a TBI is sustained, a reasonable timeframe for emergency care in both civilian or military life.

In certain embodiments, the compound (e.g., M-channel opener) is retigabine, a derivative thereof, or a pharmaceutically acceptable salt thereof. In certain embodiments, the compound (e.g., M-channel opener is RL648_81, a derivative thereof, or a pharmaceutically acceptable salt thereof.

In certain embodiments, the method further comprises reducing neuronal excitability in the brain of a subject via exposure to the M-channel opener. In certain embodiments, the method further comprises reducing cellular energy demand in a brain of the subject via exposure to the M-channel opener. In certain embodiments, administering the therapeutically effective amount of the compound prevents the development of chronic traumatic encephalopathy in a brain of the subject for at least two years following TBI.

The compound can be delivered intravenously, intramuscularly, subcutaneously, transdermally, orally, nasally or via any other administration method suitable in the medical arts for the particular subject.

In certain embodiments, the method further comprises preserving permeability of a BBB of a brain of the subject via exposure to the M-channel opener.

The step of administering the therapeutically effective amount of the compound can be performed within 1 hour of the subject experiencing the at least one TBI event, for example. In certain embodiments, administration of the therapeutically effective amount of the compound is performed within 30 minutes of the subject experiencing the at least one traumatic brain injury event.

In certain embodiments, the therapeutically effective amount of the compound is administered at between 0.3 mg/kg-3.0 mg/kg of body weight of the subject. In certain embodiments, the therapeutically effective amount of the compound is administered at 1.0 mg/kg of body weight of the subject.

The method can further comprise administering a second therapy for treatment of the TBI to the subject, where desired. In certain embodiments, the second therapy comprises stem cell therapy, hardware implantation, ultrasound therapy, or a therapeutically effective amount of a compound comprising an adenosine A₃ receptor agonist, an anticonvulsant, a coma-inducing drug, or a diuretic.

Methods of reducing a subject's susceptibility to experiencing a seizure or cognitive dysfunction following a TBI are also provided. In certain embodiments, a method of reducing a subject's susceptibility to seizure or cognitive dysfunction comprises administering a therapeutically effective amount of a compound comprising an M-channel opener to a subject who has experienced a TBI, wherein administration occurs within six hours after the TBI. In certain embodiments, the subject is experiencing or at risk of experiencing metabolic exhaustion in cells of a brain and administering the therapeutically effective amount of the compound reduces neuron hyperexcitability in a brain of the subject.

As with other methods provided herein, the compound can be retigabine, a derivative thereof, or a pharmaceutically acceptable salt thereof (or any of the other M-channel openers described herein). In certain embodiments, the therapeutically effective amount of the compound is administered at between 0.3 mg/kg-3.0 mg/kg of body weight of the subject. In at least one exemplary embodiment, administering the therapeutically effective amount of the compound to the subject prevents the occurrence of TBI-induced hypersomnia in the subject.

Methods of reducing hyperexcitability in a brain of a subject are also provided, such methods comprising administering a therapeutically effective amount of a compound comprising an M-channel opener to a subject, wherein the subject has experienced at least one TBI and the M-channel opener upregulates at least a KCNQ2 subunit, a KCNQ3 subunit, or both of an M-channel of a brain of the subject.

Currently, there are no medical treatments for either kind (blunt or blast) of TBI whatsoever, and thus, widespread mortality and morbidity numbering in the millions is the result. Thus, the present invention is the first treatment after a TBI event to prevent brain damage and dysfunction for millions of Americans and tens of millions of individuals worldwide. Thus, the present invention represents a completely new and novel approach for treatment of TBI that has been tested by no other lab or entity, worldwide.

### BRIEF DESCRIPTION OF THE DRAWINGS

FIG. 1 is a schematic of the chain of events of an embodiment of the present disclosure.

FIG. 2 depicts general characteristics of M channels as used in the present disclosure.

FIG. 3 shows graphs tracking action potential (AP) firing in response to various stimuli as used in the present disclosure.

FIGS. 4A through 4E depict graphs (FIGS. 4A, 4B, 4D, and 4E), an illustrative schematic and photograph (FIG. 4C) and electroencephalography (EEG) recordings (FIGS. 4A and 4B) documenting experimental support of the present disclosure.

FIG. 5A shows representative ex vivo images of near-infrared (NIR) fluorescent dye PSVue794 probed brains, with the probe localized to the peri-injured area.

FIG. 5B shows the data from FIG. 5A quantified in a bar graph.

FIGS. 5C and 5D show data from the blood brain barrier disruption studies, with FIG. 5C showing graphical data from four different faces of the brain, and FIG. 5D showing ex vivo images of NIR fluorescent dye probed brains.

FIGS. 6A, 6C, 6D, 6E, 6G, 6H, 6I, and 6K depicting graphs documenting experimental support of the present disclosure, FIG. 6F showing images of different cortical hemispheres of the brains from the study, and FIG. 6J showing images of labeling by the fluorescent dye, Fluoro Jade B (FJB), which was used as an indicator of TBI-induced death of neurons.

FIGS. 7A and 7B depict graphs documenting experimental support of the present disclosure.

FIG. 8 depicts a graph demonstrating dentate gyms responses to the chemoconvulsant, kainic acid, between sham, TBI only and TBI+RL648_81-treated mouse groups of the present disclosure.

FIG. 9 depicts in vivo imaging of intracellular calcium levels in living brain nerve cells in vivo, as a reporter of activity/hyperactivity and quantified graphical data from the same study.

FIG. 10 shows graphs and in vivo images recording the optical measurement in living mice in vivo of extracellular glutamate [glutamate]ₒ by viral expression of a fluorescent sensor in support of the present invention.

FIG. 11 shows a graphical representation of the long-term effects of TBI, wholly distinct from treating seizures with anti-convulsants, showing the probability of seizures for different groups of mice tested using the present invention.

FIG. 12A shows a graphical representation of seizure duration of two groups of mice subjected to repetitive blast traumatic brain injury (rbTBI) protocol (i.e. three directed head-on blast TBI with 24 hour intervals between each injury, which mimics mild blasts equivalent to shockwaves experienced during combat related to the firing of large-caliber weapons from comrade troops), the first group labeled “Blast” receiving an intraperitoneal (i.p.) injection of only vehicle 30 minutes after each blast, and the second group labeled “Blast+RTG” receiving one i.p. injection of RTG 30 minutes after each blast, wherein mice were video and electroencephalogram (EEG) monitored from the second to the fourth day after the TBIs (72 hours in total).

FIG. 12B shows representative EEG recordings from the mice in the study of FIG. 12A, with those acutely treated post-TBI either with vehicle only (left, labeled “Blast”) or RTG (right, labeled “Blast+RTG” with seizure events marked by the black, horizontal line over the chart.

FIG. 13A shows a graphical representation of the number of seizures experienced in the tested cohorts from the first month to the ninth month post treatment. FIG. 13B shows representative EEG recordings from mice in the study of FIG. 13A with those acutely treated post-TBI either with vehicle only (“Blast”) or RTG (“Blast+RTG”), with seizure events marked by the black, horizontal line below the Blast chart (notably, the Blast+RTG chart does not report any seizure events).

FIG. 14A shows data from a component analysis of EEG recording data, identifying slow wave sleep (SWS), rapid eye movement sleep (REM sleep), and awake state (WK) in the mice from the rbTBI study taken the first week post-TBI. FIGS. 14B-14E show graphical data representative of the same analysis displayed in FIG. 14A, but presented as precent of total time in the identified states in the first week post-TBI, with “US” representing transition/undetermined state.

FIG. 15A shows EEG recording data and FIGS. 15B-15D show graphical data related to gamma and delta cortical waves measured during slow wave sleep in mice that underwent the rbTBI study, observed 2 days after the last TBI treatment/injury (S1) and 9 months after the last TBI treatment/injury (S9).

FIG. 16A shows summarized immunoblot data of TAR DNA-binding protein 43 (TDP-43) expression in the cerebral cortex, normalized to beta-actin (β-actin) expression and FIG. 16B shows phosphorylation levels of TDP-43 in the cerebral cortex following acute RTG treatment administered after multiple mild blast TBIs.

FIG. 16C shows micrographs of mouse cortex brain slices immunostained for TDP-43 (first column on the left), NeuN as a neuronal marker (second column from the left), and DAPI to label nuclei (third column from the left), with the far-right column displaying merged images of the other columns.

FIGS. 16D and 16E show graphical data representative of the mean fluorescence (FIG. 16D) and maximum fluorescence (FIG. 16E) intensity of TDP-43 in each of the samples.

FIGS. 17A-17D show data measured post-TBI treatment after multiple mild blast TBIs, with FIGS. 17A and 17C showing micrograph of nerve fibers of samples taken from a Sham cohort, a Blast cohort, and a Blast+RTG cohort from the corpus callosum (FIG. 17A) and the striatum (FIG. 17C) of mice of about two years old, in which myelin is labeled with Luxol fast-blue staining, and FIGS. 17B and 17D graphically representing the data shown in FIGS. 17A and 17C, respectively.

FIGS. 18A show images of hippocampal CA1 slices taken from all three study cohorts (Sham, TBI, and TBI+RTG), with the arrows indicating examples of neurons counted as c-Fos positive.

FIG. 18B shows a graphical representation summarizing the c-Fos expression exhibited in FIGS. 18A, with the bars summarizing increased c-Fos expression that was blunted by acute RTG treatment.

FIGS. 19A and 19B show graphical data related to the occurrence of spontaneous myoclonic seizures in mice after one blunt TBI (CCCI) (FIG. 19A) and the occurrence of clonic or tonic-clonic seizures following a normally sub-seizure threshold dose of the chemoconvulsive agent, pentylenetetrazol (PTZ; 35 mg/kg; given via subcutaneous (s.c.) injection) (FIG. 19B), with numbers of mice displaying seizures or PTZ-induced seizures and the total N of mice per group given above each bar (Sham mice (N=7) did not show spontaneous or PTZ-induced seizures and RTG and RL648_81 treatments were injected in the relevant cohorts once 30 minutes post-TBI (i.p.).

FIG. 20A shows data from in vivo two-photon confocal microscopy taken through a “glass window” over mouse cortex and FIG. 20B shows a graphical representation summarizing the data of FIG. 20A.

FIG. 21 shows graphical data representative of optimizing dosage of RTG and/or treatment effects in mice following one blunt TBI (CCCI model).

### DETAILED DESCRIPTION

For the purposes of promoting an understanding of the principles of the present disclosure, reference will now be made to the embodiments illustrated in the drawings, tables, and figures and specific language will be used to describe the same. It will nevertheless be understood that no limitation of the scope of this disclosure is thereby intended.

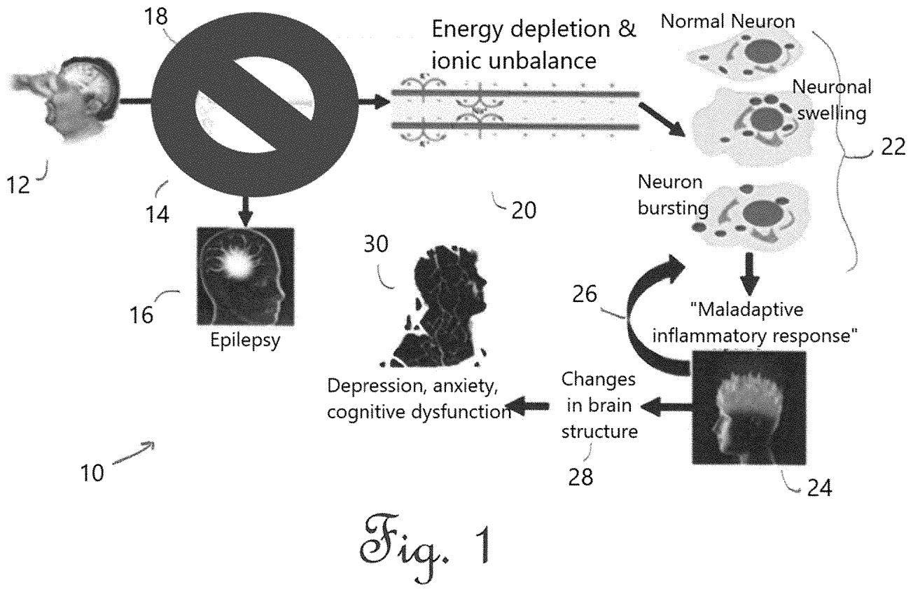

FIG. 1 depicts a chain of events 10 associated with a traumatic brain injury (“TBI”). TBI can result from an external impact to the head (the impact of a fist or blunt instrument to the front of someone's head 12, for example). Whereas TBI is commonly thought of as resulting from a severe blow to the head, it is important to note that it can also result from shockwaves or blast exposure. Blast-induced TBI, a leading cause of combat and training TBI among military personnel, can result from explosive blast overexposure, or from the shockwave from large-caliber weapons as examples, both of which generating shockwave energy that impacts the head and the brain. In general, four types of blast injury have been described in relation to deployed service personnel: primary blast injuries (relating to the over-pressure, also termed shockwave); secondary blast injury (relating to primary and secondary fragments); tertiary blast injuries (relating to blast wind, also termed dynamic over-pressure); and quaternary blast injuries (relating to other injury mechanisms).

TBI is a complicated pathological process that can result in different brain injury patterns of variable clinical severity, ranging from mild, moderate, to severe. A concussion (mild TBI), brain contusions (moderate TBI), and penetrating brain injuries (severe TBI) are all examples of primary insults associated with TBIs of varying severity. Post concussive syndromes involving significant, persistent headaches and blurred vision, and short-term memory loss also often occur.

In addition to the primary insults, TBI can cause secondary insults, often characterized by a set of biochemical cascades. Post-TBI, excessive neuronal firing 14 commences. If allowed to continue untreated, post-traumatic seizures and epilepsy 16 can result. The imbalance between a higher energy demand for repair of cell damage and decreased energy production led by mitochondrial dysfunction in neurons can aggravate cell damage. Indeed, if excessive neuronal firing 14 does not cease, next follows the depletion of intracellular energy stores and ionic imbalance 20, which can continue until healthy neurons begin to swell and ultimately burst (lyse), as shown in event 22 in FIG. 1. A maladaptive inflammatory response 24 can also occur, in response to such cell death. From here, further swelling and bursting of neurons can continue, as shown by return loop 26. Changes in the brain structure and proper functioning 28 can become evident, leading to the final event 30 of depression, anxiety, locomotor defects and cognitive dysfunction, such as deficits in attention, verbal and nonverbal learning, short term and working memory, visual and auditory processing, problem solving, mood disorders, cerebral processing speed and motor functioning.

Methods of treatment for brain damage or cognitive dysfunction (e.g., resulting from one or more TBIs) are provided. In certain embodiments, methods for reducing seizure susceptibility in a subject (or reducing a subject's susceptibility to experiencing dysfunction) having suffered a TBI are provided. Still further, methods for reducing hyperexcitability in a brain of a subject are provided. The methods provided herein can be used in the treatment of a subject after experiencing any TBI, irrespective of the severity thereof or if the TBI was caused by blunt force or is blast induced.

Such methods comprise administering a therapeutically effective amount of a compound that reduces neuronal excitability and/or cellular energy demand via M-current enhancement. Such methods can, in certain embodiments, be administered to a subject immediately following a TBI, for example, within 30 minutes or one-hour post-TBI. In certain embodiments, the therapeutically effective amount of the compound is administered acutely (e.g., immediately post-TBI, within 10 minutes post-TBI, within 15 minutes post-TBI, within 30 minutes post-TBI, within 35 minutes post-TBI, within 40 minutes post-TBI, within 45 minutes post-TBI, or within 1 hour post-TBI). In human subjects, this time window can be longer (e.g., 2 hours, 3 hours, 4 hours, 5 hours or more).

In certain embodiments, the compound is an M-channel opener. Such an M-channel opener can, when administered to a subject, for example, upregulate at least a KCNQ2 subunit, a KCNQ3 subunit, or both of an M-channel of a brain of the subject (described in additional detail below).

In certain embodiments, the compound is retigabine:

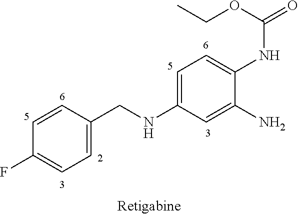 [SMILES: CCOC(=O)Nc1ccc(NCc2ccc(F)cc2)cc1N]

or a derivative thereof, or a pharmaceutically acceptable salt thereof.

In certain embodiments, the compound is selected from the group consisting of (2-amino-4-(4-fluorobenzylamino)-1-ethoxycarbonylaminobenzene, an aminoalkyl thiazole derivative (known as Ezogabine in Europe and sold in the USA under the trade name, Potiga, formerly FDA approved but now off the market as an anti-convulsant), ICA-069673 ([N-(2-chloro-5-pyrimidinyl)-3,4-difluorobenzamide]) and ICA-27243 and the derivatives of each of the foregoing, (2-amino-4-(arylamino-phenyl) carbamates (SF0034, RL648_81) and derivatives thereof, and other such “openers” (agonists) of KCNQ2-5 potassium ion channels that increase their currents in all known neurons of the central and peripheral nervous systems.

In certain embodiments, the compound is RL648_81 having the structure:

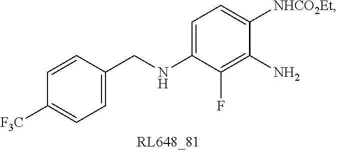

or a derivative thereof, or a pharmaceutically acceptable salt thereof. As shown above, RL648_81 is a fluorinated analog of retigabine. It will be appreciated that the compound can comprise other M-channel openers, for example other analogs of retigabine, provided such analogs can have an activating/upregulating effect on at least KCNQ2/3 channel currents when administered to a subject.

As used herein, the terms “patient,” “subject,” and “individual” are used interchangeably. None of the terms require the supervision of medical personnel. For example, administering to an individual includes the individual administering the therapeutic agent to themselves, as well as a medical professional administering the therapeutic agent to the individual.

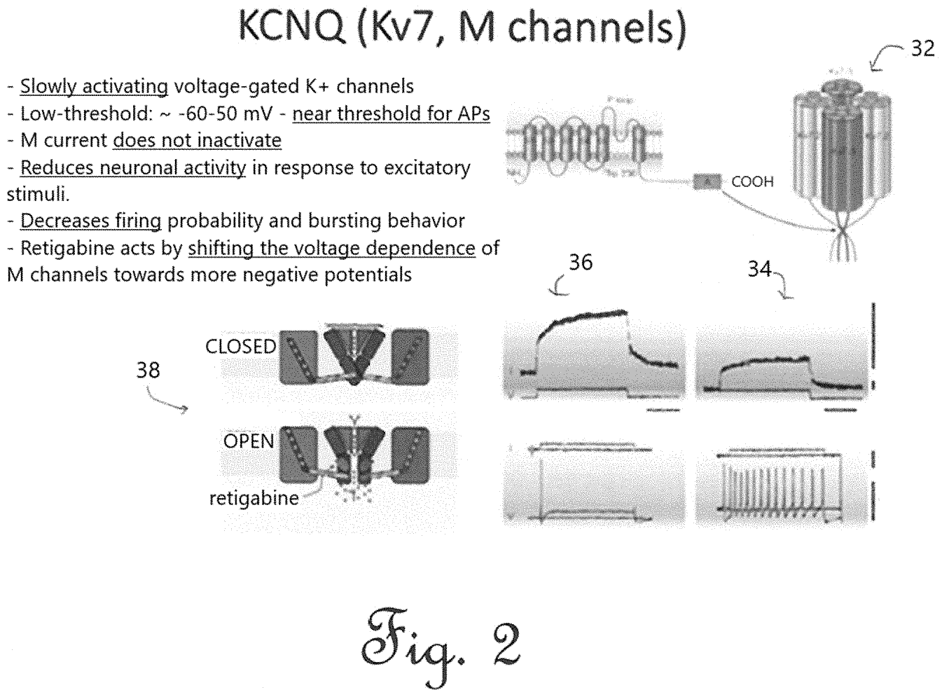

Referring now to FIG. 2 the general characteristics of M-channels 32 are shown. M-channels 32 are voltage-gated K⁺ channels with prominent expression throughout the brain that are important in raising the threshold for firing an action potential (AP). M-channels 32 are tetramers of four KCNQ subunits 2-5 that can be homomeric or heteromeric, depending on their subtype. Most M-channels in neurons comprise KCNQ2 & KCNQ3 (Kv7.2 & Kv7.3) subunits with varying stoichiometries governed only by their relative expression, but homomers can form as well. Other subtypes include KCNQ4 (found in the inner ear and smooth muscle and typically expressed as homomers) and KCNQ5 (found in some areas of the brain and also in smooth muscle). KCNQ3 is promiscuous and can form either homomers or heteromers with KCNQ2, KCNQ4, or KCNQ5.

M-channels 32 play dominant roles in control over excitability of neurons and are thus implicated in myriad neurological and psychiatric disorders. They are unique in that they open at rest and are even more likely to be open during depolarization. Conversely, when the muscarinic acetylcholine receptor (MAChR) is activated (e.g. in sympathetic neurons), the channel closes. FIG. 2 shows current waveforms 34 and 36 before and during stimulation of mAChR (top) 34 and the greatly induced (AP firing 36 when M current is reduced as shown.

These slowly activating voltage-gated K⁺ channels have a low threshold of about −60 to −50 mV—near the threshold for AP generation. The M-type K⁺ current 32 does not inactivate, unlike most other voltage-gated K⁺ currents, but instead reduces neuronal activity in response to maintained excitatory stimuli which thus limiting continued firing probability and bursting behavior. Retigabine and similar M-channel “openers,” act by shifting the voltage dependence of M-channels towards more negative potentials, as shown in representation 38 of FIG. 2. Accordingly, downregulation of such M-channels has been implicated in several hyperexcitability-related disorders including epilepsy, neuropathic pain, and tinnitus.

Turning now to FIG. 3, the role 40 of M-type K⁺ channels in control of neuronal excitability is described. M-type K⁺ current is underlied by KCNQ2-5 (Kv7.2-7.5) K⁺ channel subunits, as depicted by representation 42. Various currents are applied in “current-clamp” mode 44 ranging from 50 pA to −100 pA, and the resulting AP firing recorded.

Activators of these M-channels 32 reduce the excitability of central and peripheral neurons. Graph 48 shows APs in response to stimuli in a sympathetic neuron when M current is enhanced by the “opener” drug, retigabine. Complete silencing of the neuron occurs, as shown by graph 48. When M channels are completely blocked by the prototype M-channel “blocker,” leopardine, the result is out-of-control AP firing as when a seizure occurs, conversely, when retigabine is applied to a “brain slice” of the hippocampus in the temporal lobe (the brain lobe most prone to seizures and resultant epilepsy), as shown by graphs 52, in which the rapidly-firing neuron is converted into one totally quiescent. Thus, manipulation of M-channel activity profoundly affects excitability, and a pharmacological “opener” can act as a potent brake on detrimental hyperexcitability.

Acutely reducing neuronal excitability and cellular energy demand via M-current enhancement is a novel model of therapeutic intervention against post-TBI brain damage and dysfunction. This is done by reducing the initial TBI-induced hyperexcitability before seizures occur that only exacerbate cellular energy depletion, thus, “nipping in the bud” the deleterious chain of events that cause a TBI to be so damaging to the brain (see, e.g., FIG. 1).

As discussed further below, the newer M-channel opener, RL648_81, applied 30 minutes after a blunt TBI, reduces the increased seizure susceptibility in brain slices, ex vivo. RL-648_81 also prevents TBI-induced hyper-excitability in cortex when neuronal activity is imaged in vivo through a cranial window in a living mouse brain. Finally, experimentation demonstrates that brain damage manifesting in long-term epilepsy occurs after 3-consecutive mild-moderate blast or blunt TBIs, and that such is prevented by M-channel augmentation, a series of events often experienced by members of the armed services or as a result of chronic domestic abuse/violence against women, or by those participating in many common contact sports around the world. Administration of an M-channel opener once after each mild TBI will abolish the deleterious effects on the brain.

In experiments, the present invention modeled mild blast TBI injury in mice to observe effects on neuronal excitability. Mice were exposed to an average of 14.6±0.5 psi (100±3 kPa) maximum peak overpressure, which has been classified as mild blast TBI (0-145 kPa) in rats and mice using a number of physiological parameters. Experiments were first performed to evaluate the effect of the blast TBI model, with a single exposure, or with repeated second and third exposures using 24-hour intervals between TBI.

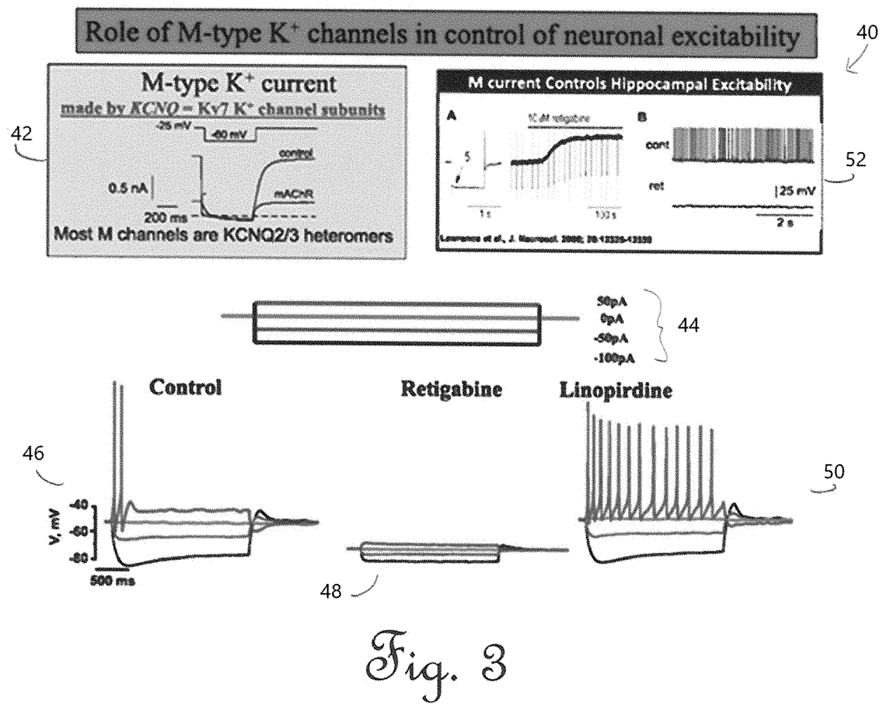

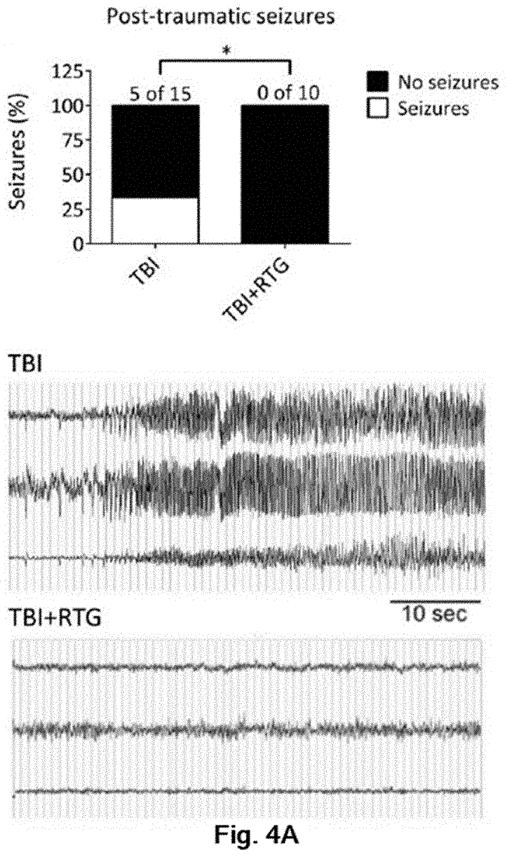

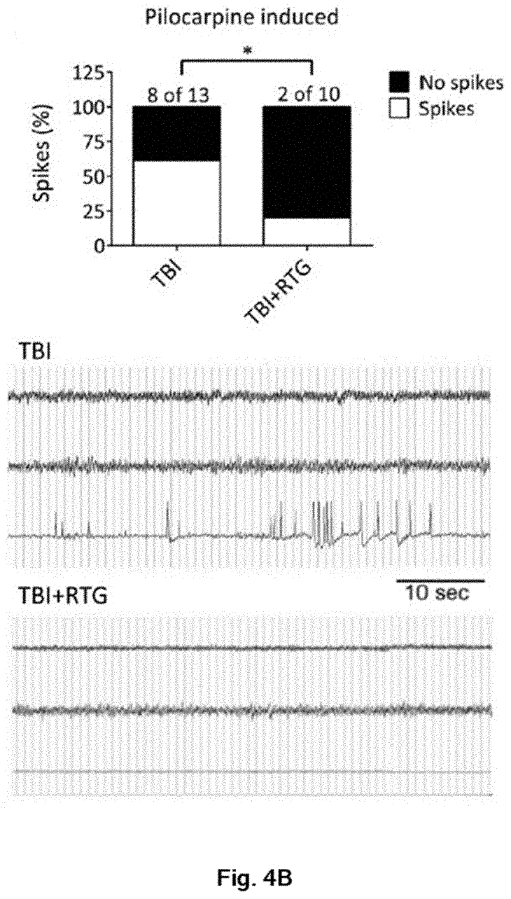

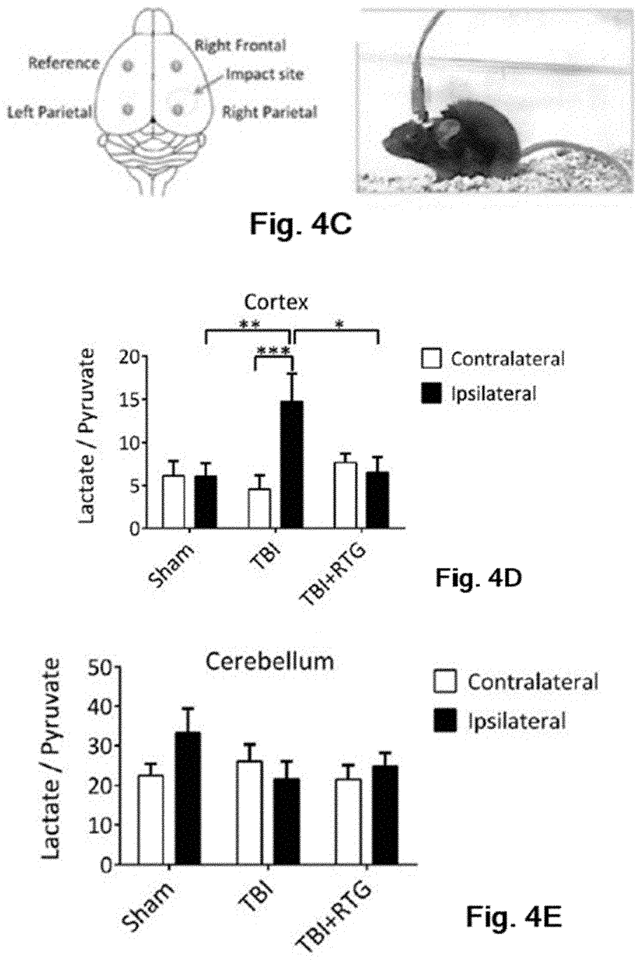

Referring now to FIGS. 4A through 4E, as an indicator of blunt TBI-induced seizures and TBI-induced seizure susceptibility, mice were continually monitored by video and EEG. The present invention shows that, whereas mice subjected to TBI and no drug, 33% of mice displayed post-traumatic seizures, of those mice subjected to TBI and administered the M-channel opener, retigabine (RTG), 30 minutes post-TBI, zero mice displayed post-traumatic seizures, as shown in FIG. 4A. In addition, whereas mice subjected to TBI and no drug, as an indicator of cellular metabolic stress, the lactate/pyruvate ratios in at least the cortex were increased by nearly 3-fold in the same side of brain as the trauma, whereas those receiving RTG 30 minutes post-TBI experienced no significant increase in cellular metabolic stress as compared to the Sham cohort (control), as shown in FIG. 4D, respectively. Accordingly, whereas the data support CCCI to cause metabolic exhaustion in cells of the brain hemisphere ipsilateral to the site of TBI, it was prevented by acute post-TBI administration of an M-channel opener such as RTG.

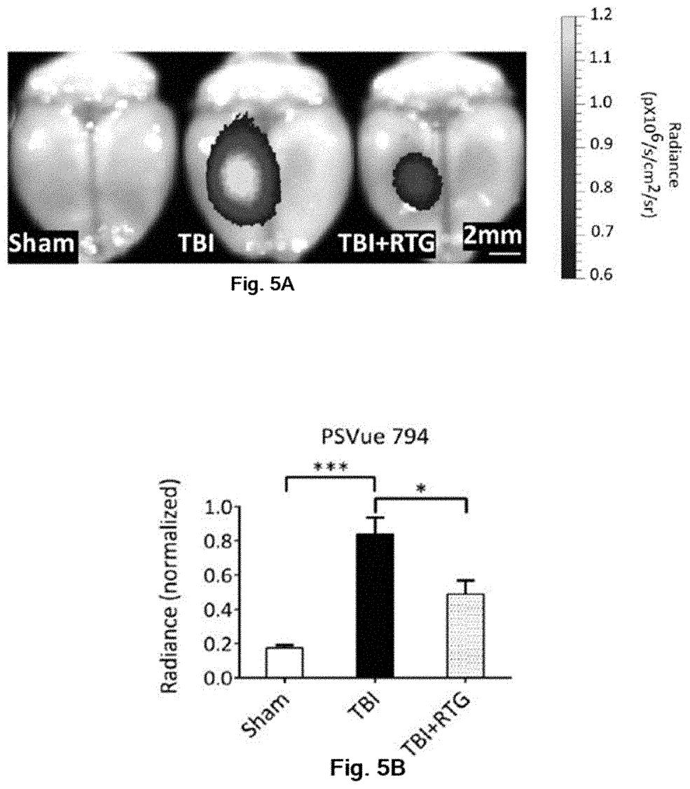

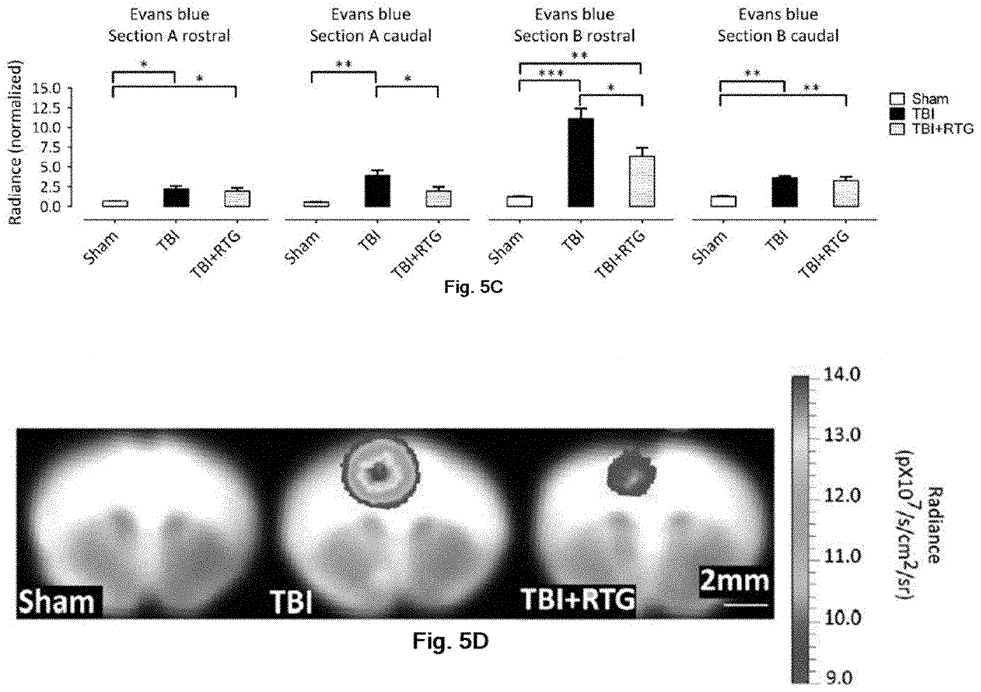

Referring now to FIGS. 5A-D, as an indicator of breakdown of the Blood-Brain Barrier (BBB), the Evans Blue extravasation assay at 72-hour post-TBI showed a dramatic breakdown of the BBB in mice not treated with any drug, but in mice subjected to TBI and treated with RTG 30 minutes post-TBI, the breakdown of the BBB was reduced by about 50%, as shown in FIG. 5C. These results, taken with the PSVue794 characterization shown in FIGS. 5A and 5B, support significant neuroprotection by M-current augmentation through administration of RTG.

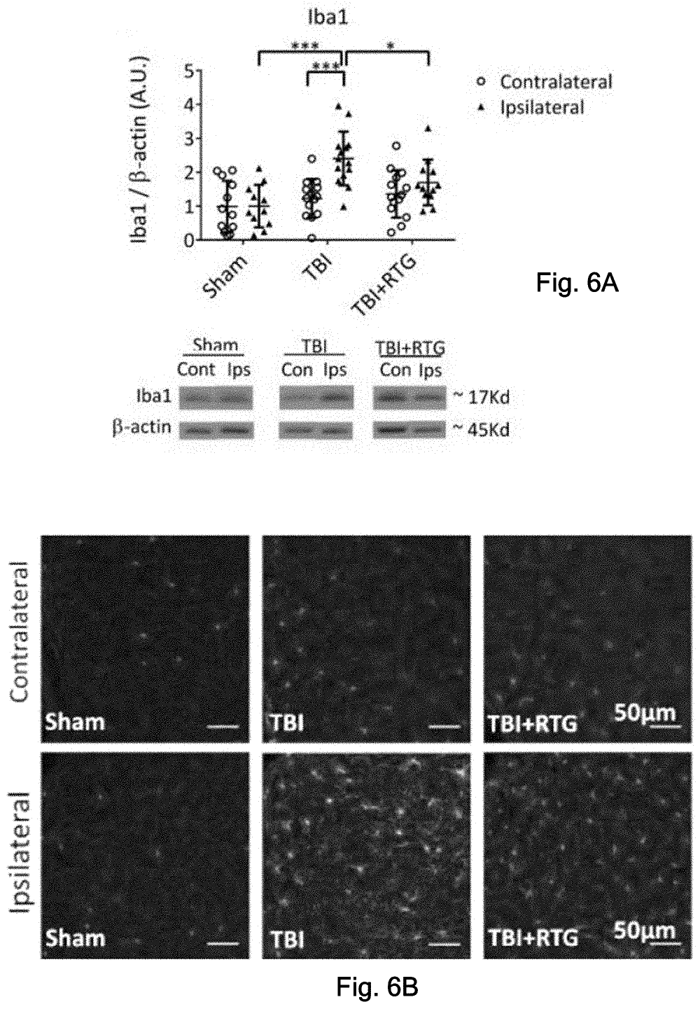

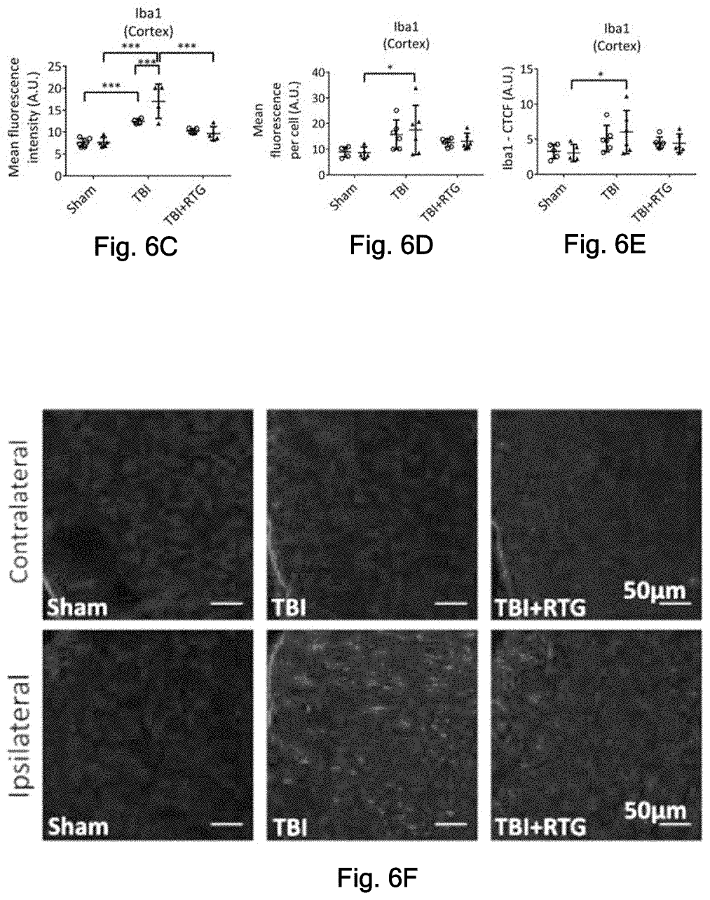

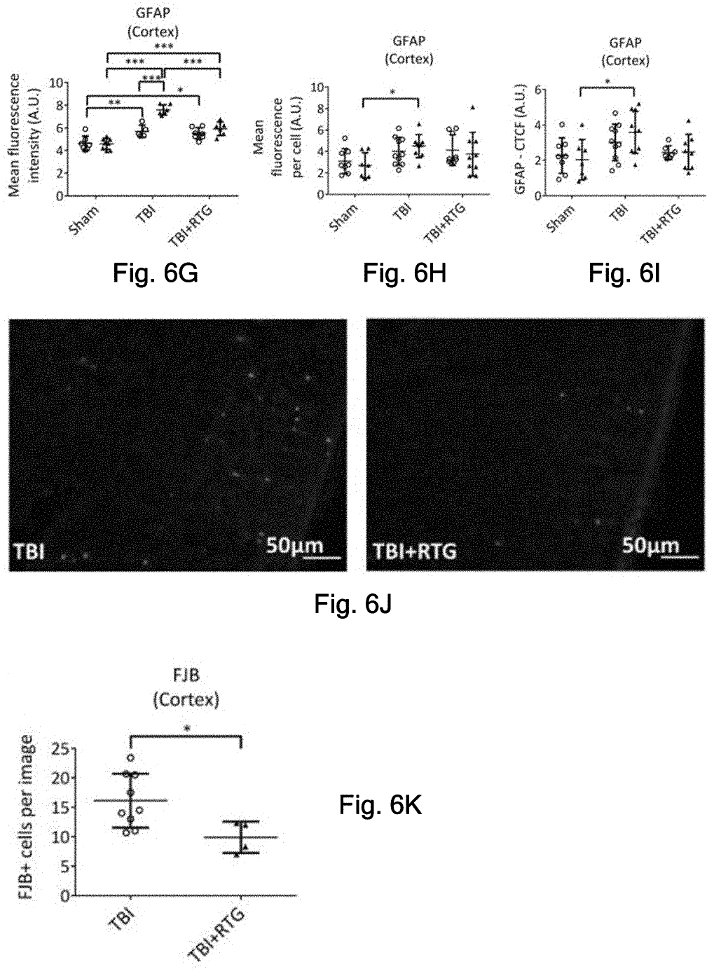

The following four sections concern the blockade of post-TBI mal-adaptive immunological/inflammatory responses block by augmentation of M current following TBI. Referring now to FIGS. 6A-C, as an indicator of microgliosis, the biomarker ionized Ca²⁺ binding adaptor molecule 1 (Iba-1) was assayed, both by western blot and by immunofluorescence. In mice subjected to TBI but no drug, Iba1 expression was increased by about double on the ipsilateral side of the brain to the trauma, but in mice subjected to TBI and RTG within 30 minutes of trauma, there was no such increase in either type of Iba1 assay. This supports that while a significant increase in microglial/macrophage hyper-activation was observed after TBI, it was prevented by RTG treatment. Indeed, M-current augmentation suppressed the TBI-induced microglial activation in the ipsilateral cortex (see FIG. 6A).

FIGS. 6A through 6K demonstrate M-current augmentation suppressed TBI-induced microglial activation in the ipsilateral cortex. With specific reference now to FIGS. 6F-I, as an indicator of TBI-induced astrogliosis, the levels of glial fibrillary acidic protein (GFAP) were assayed after TBI by immunofluorescence from anti-GFAP antibodies. In mice subjected to TBI but did not receive a drug, GFAP levels were increased by nearly double in cortex on both sides of the brain, but in mice that received RTG 30 minutes post-TBI, there was no such increase.

Referring now to FIG. 6J, labeling by the fluorescent dye, Fluoro Jade B (FJB), was used as an indicator of TBI-induced death of neurons. In mice subjected to TBI but not drug, there was substantial labeling of neurons throughout the brain, indicating substantial TBI-induced death of neurons. But in mice subjected to TBI and administered RTG 30 minutes post-TBI, labeling of brain neurons by FJB was minimal, as shown in FIG. 6K. This supports that administration of RTG within 30 minutes of TBI decreased neuronal death.

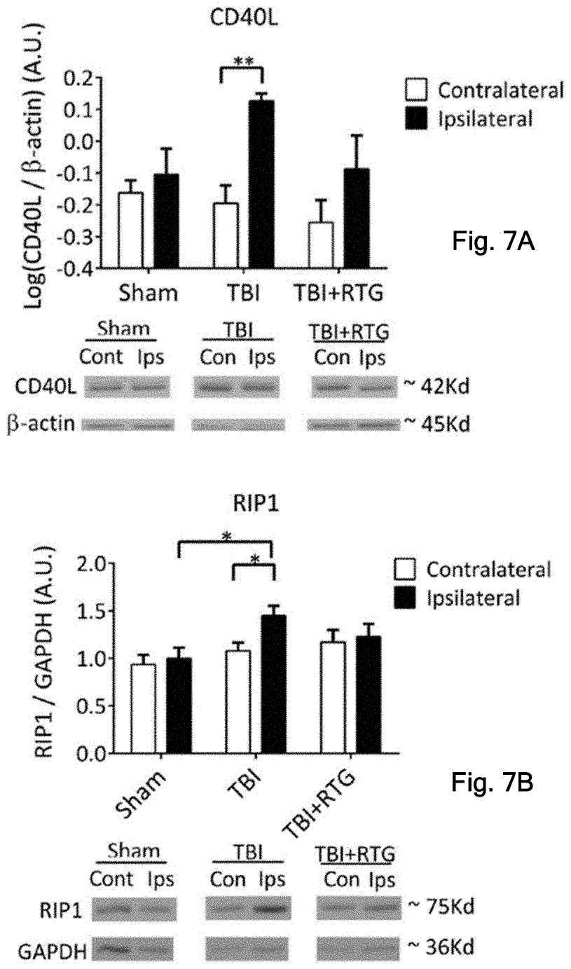

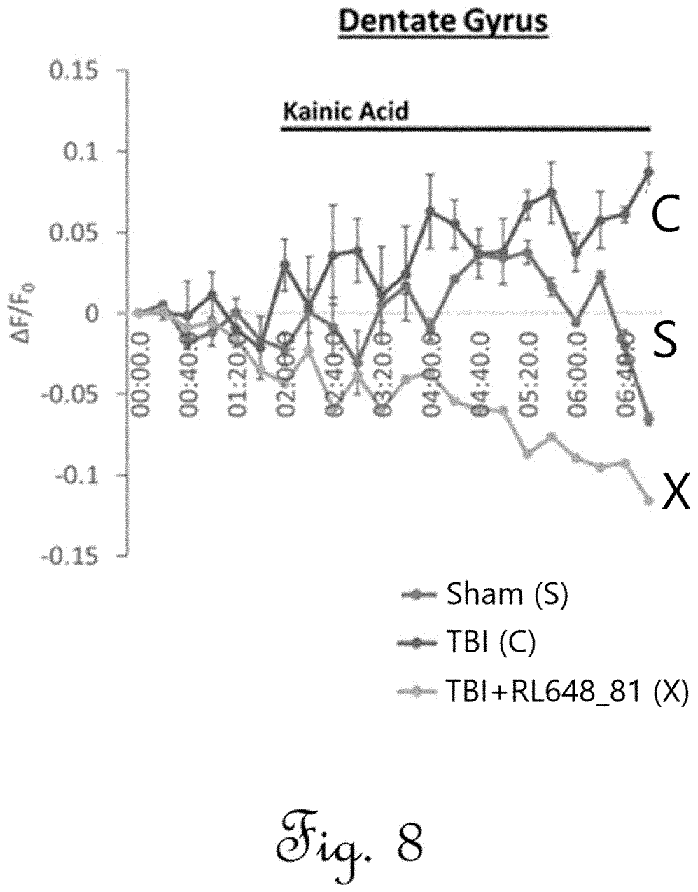

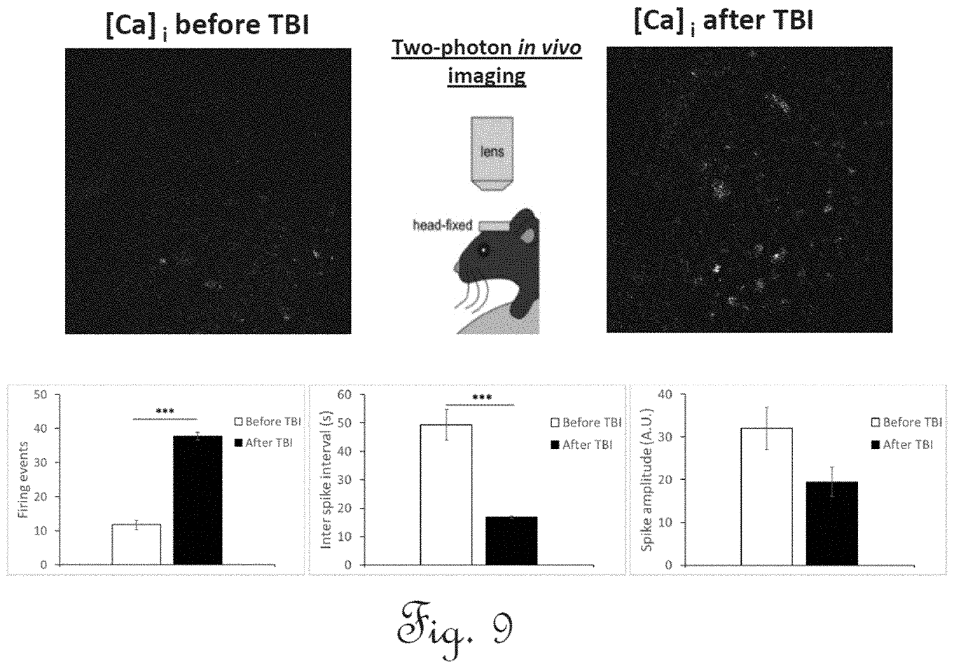

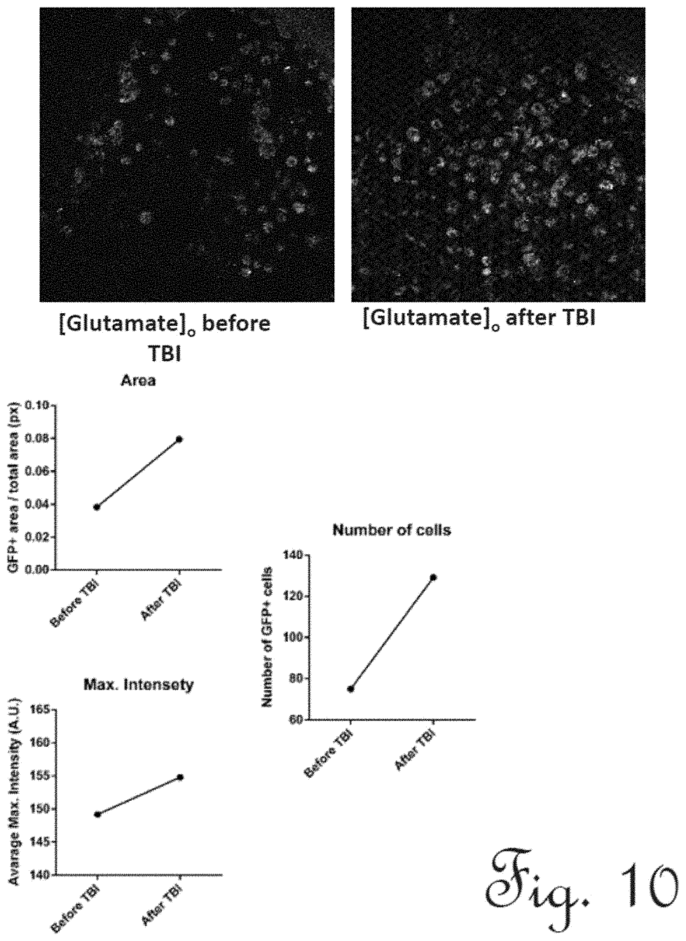

Referring now to FIG. 7A, as an assay of the mal-adaptive immune response using the inflammatory biomarker, CD40 ligand (CD40L), mice subjected to a blunt TBI but no drug exhibited over a two-fold increase in levels of CD40L over that of sham mice or mice subjected to TBI but not drug However, in mice subjected to a blunt TBI followed after 30 minutes by RTG, the levels of CD40L were not significantly different than in sham mice. This data supports that acute augmentation of M current after TBI (e.g., through administration of a therapeutically effective amount of a compound such as RTG, a derivative or analog thereof (including, without limitation, RL648_81)) reduces the deleterious inflammatory/immunological responses to injury.

Referring now to FIG. 7B, as an assay of necrosis and apoptosis of neurons, levels of the biomarker, receptor-interacting protein 1 (RIP-1), in brain were measured. In mice subjected to TBI but not drug, there was a 50% increase of RIP-1 levels in brain, indicating substantial TBI-induced necrosis and apoptosis. But in mice administered RTG 30 minutes post-TBI, the levels of RIP-1 in brain were not significantly different than shams. This supports that TBI provokes inflammatory/immunological responses that results in the production of cell-death-related proteins. Further, the inflammatory response and cell death are correlated, supporting an etiology of traumatic cerebral damage.

In another embodiment of the present invention, ex vivo imaging of intracellular calcium levels in neurons, which are a reporter of activity or hyper-activity of neurons, were assayed in the brain slices using transgenic mice expressing the genetically encoded indicator of calcium levels, GCaMP6f expressed only in neurons. As seen in FIG. 7, such transgenic mice were used to image intracellular calcium levels inside critical subsets of brain neurons using ex vivo brain-slice imaging experiments.

Turning now to FIG. 8, the chemo-convulsant, kainic acid (KA), was used to stimulate neurons from brain slices of the dentate gyms (DG) of the hippocampus (the part of the brain where most new memories are thought to form) from sham mice, mice subjected to a TBI but not drug or TBI followed by RL-648_81. RL648_81 is a new generation drug that facilitates M-current solely produced by heteromeric tetramers of KCNQ2&3 subunits, the subunits found predominantly in brain neurons, thus sparing side-effects of RTG on smooth muscle, such as that in bladder, which causes urinary incontinence.

Using this technique, it was discovered that M-current augmentation by the administration of RL648_81 occludes TBI-induced increased hyperexcitability in response to KA stimulation. The experiments show the hippocampus to have a stronger TBI-induced increase in hyper-activity in response to KA stimulation, which was completely abolished by RL648_81 treatment 30 minutes after the TBI. Thus, the present invention concerns not only one molecule, but rather the general approach of preventing TBI-induced brain damage, which has never before been postulated or demonstrated.

Referring now to FIG. 9, another embodiment of the present invention concerns in vivo imaging of intracellular calcium levels in neurons from living brain in vivo, as a reporter of activity/hyperactivity. The genetically-encoded calcium reporter, GCaMP6f, was used in transgenic mice in which the reporter is expressed in critical subsets of brain neurons, allowing in vivo recordings of neurons of living brain, as a reporter of activity/hyperactivity in cortex induced by TBI, the first event in the hypothesis of the present invention, which causes TBI-induced brain damage (See, e.g., FIG. 1). For these experiments two-photon confocal live imaging of mouse cortical neurons through a cortical window was used. Live video records of the activity of cortical neurons in mice before and after TBI were made. As can be seen in the graphs presented in FIG. 9, a significant increase in neuronal firing and a decrease in inter spike (inter AP) interval was observed after a blunt TBI.

Another embodiment of the present invention, and referring now to FIG. 10, concerns the optical measurement in living mice in vivo of extracellular glutamate [glutamate]ₒ, the major excitatory neurotransmitter in the brain, by pseudoviral expression of a fluorescent sensor, iGluSnFR packaged in the viral vector AAV-PHP.B-Syn1, injected i.v. into the mice. Again, this sensor as designed herein can only be expressed in brain neurons, as was the case for GCaMP6s/f. It can be seen that in mice subjected to a TBI, [glutamate]ₒ is strongly increased to abnormal levels. These results support the hypothesis of the present invention regarding TBI-induced hyper-excitability as the first event in the cascade of events resulting in brain damage and dysfunction.

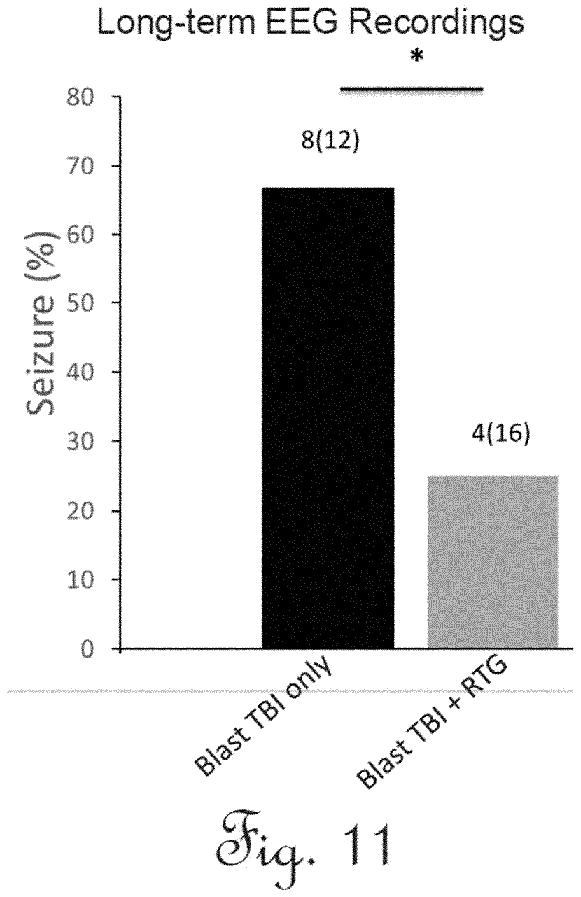

In another embodiment of the present invention, and now referring to FIG. 11, the long-term effects of TBI are shown, in particular the development of epilepsy disease, which is wholly distinct from treating seizures with anti-convulsants. The present disclosure also addresses the case of repetitive, moderate, blast TBIs, such as those experienced routinely by members of the armed forces. Mice were subjected to three blast TBIs, one day apart, either with no drug application, or with application of RTG 30 min after each one. As can be seen, 67% of mice displayed spontaneous seizures over a long period of time, as observed by electroencephalogram (EEG) and video recording from the period of 1 month to 12 months after injury. The occurrence of these seizures confirms the occurrence of post-traumatic epilepsy (PTE) after repetitive blast-TBI. RTG treatment 30 minutes after each blast TBI was able to significantly reduce PTE occurrence many months to a year later. Only 25% of the RTG treated mice developed PTE. Eight sham animals were also monitored and no seizures were observed. There is currently no FDA-approved treatment for PTE. Hence, treatment with M-channel openers represents a paradigm change in PTE prevention.

Brain damage, deficits and dysfunction resulting from commonly occurring TBIs can be prevented by acute administration of an M-channel opener shortly after the occurrence of a TBI. The TBI can be of the blunt type or the blast/shockwave type, as commonly experienced in falls, impact of the head with a hard object, vehicular accidents, explosions on the battlefield in the vicinity of military service personnel, or shock waves from the use of large weapons near the victim by either a fellow service member, or the enemy. Even very mild TBIs, although not perceptible to the victim, when repeated multiple times, cause brain damage, which can also be prevented by M-channel pharmacological openers when administered after each TBI, or after a succession of TBIs.

In certain embodiments, the methods hereof can comprise reducing neuronal excitability in a brain of a subject via exposure to the therapeutically effective amount of the compound (e.g., an M-channel opener such as retigabine or a derivative, analog, or pharmaceutically acceptable salt of any of the foregoing). This is important as neuronal hyperexcitability can result in the subject experiencing (or at risk for experiencing) metabolic exhaustion in cells of the brain, which can lead to seizures and other adverse effects. Accordingly, the methods hereof can include reducing a subject's susceptibility to experiencing a seizure or dysfunction of the brain following a TBI by administering a therapeutically effective amount of a compound hereof. In at least one exemplary embodiment, the compound is administered within 6 hours of the TBI (e.g., within 5 hours post-TBI, within 4 hours post-TBI, within 3 hours post-TBI, within 2 hours post-TBI, within 1 hour post-TBI, or within 30 minutes post-TBI).

In certain embodiments, the method can further comprise reducing cellular energy demand via exposure to the therapeutically effective amount of the compound. Still further, in certain embodiments and as supported by the Examples below, administering the therapeutically effective amount of the compound can prevent development of chronic traumatic encephalopathy in the brain of the subject. In some cases, this preventative effect can last up to (or over) two years following the TBI.

The method can further comprise preserving the permeability of a blood brain barrier of the subject's brain via exposure to the therapeutically effective amount of the compounds hereof.

The present invention has application in the treatment of traumatic brain injuries caused as a result of engaging in contact sports/athletic events, in which head impacts are common, including American football, soccer, ice hockey, boxing, bicycle falls and the like. Other applications in the treatment methods hereof can be to victims of violence (e.g., domestic violence, blunt trauma, or individuals experiencing Shaken Baby syndrome). Further, the present methods have applications with military personnel who are exposed to blast shockwaves. It will be appreciated that the methods hereof are applicable to treat TBI and prevent adverse results thereof, irrespective of the cause or severity of the TBI.

The various embodiments described herein may be used singularly or in conjunction with other similar methodologies. The present disclosure includes preferred or illustrative embodiments in which a method for the prevention of brain damage and dysfunction and/or hyperexcitability in a brain after blunt or shockwave blast types of TBI by a single systemic dose application (i.v. or i.p.) of an M-channel pharmacological “opener” in a brain is described. The present disclosure is intended to cover the entire spectrum of M-channel “openers,” including those listed herein, and any new compounds or derivatives of existing compounds that may be developed on this same target.

Alternative embodiments of such a method can be used in carrying out the invention as claimed and such alternative embodiments are limited only by the claims themselves. Other aspects and advantages of the present invention may be obtained from a study of this disclosure and the drawings, along with the appended claims.

The compounds can be formulated into a pharmaceutical composition. Compounds and/or compositions described herein may be administered in unit dosage forms and/or compositions containing one or more pharmaceutically acceptable carriers, adjuvants, diluents, excipients, and/or vehicles, and combinations thereof.

The term “administering” generally refers to any and all means of introducing compounds described herein to the host subject including, but not limited to, by intravenous, intramuscular, subcutaneous, transdermal, oral, nasal, and like routes of administration.

Administration of the compounds as salts can be appropriate. Examples of acceptable salts include, without limitation, alkali metal (for example, sodium, potassium or lithium) or alkaline earth metals (for example, calcium) salts; however, any salt that is generally non-toxic and effective when administered to the subject being treated is acceptable. In at least one embodiment, the salt can be ammonium acetate salt. Similarly, “pharmaceutically-acceptable salt” refers to those salts with counter ions which may be used in pharmaceuticals. Such salts may include, without limitation: (1) acid addition salts, which can be obtained by reaction of the free base of the parent compound with inorganic acids, such as hydrochloric acid, hydrobromic acid, nitric acid, phosphoric acid, sulfuric acid, perchloric acid, and the like, or with organic acids, such as acetic acid, oxalic acid, (D) or (L) malic acid, maleic acid, methane sulfonic acid, ethanesulfonic acid, p-toluenesulfonic acid, salicylic acid, tartaric acid, citric acid, succinic acid, malonic acid, and the like; or (2) salts formed when an acidic proton present in the parent compound either is replaced by a metal ion, e.g., an alkali metal ion, an alkaline earth ion, or an aluminum ion, or coordinates with an organic base, such as ethanolamine, diethanolamine, triethanolamine, trimethamine, N-methylglucamine, and the like. Pharmaceutically acceptable salts are well-known to those skilled in the art, and any such pharmaceutically acceptable salts are contemplated.

Acceptable salts can be obtained using standard procedures known in the art, including (without limitation) reacting a sufficiently acidic compound with a suitable base, affording a physiologically acceptable anion. Suitable acid addition salts are formed from acids that form non-toxic salts. Illustrative, albeit nonlimiting, examples include the acetate, aspartate, benzoate, besylate, bicarbonate/carbonate, bisulphate/sulphate, borate, camsylate, citrate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, saccharate, stearate, succinate, tartrate, tosylate and trifluoroacetate salts. Suitable base salts of the compounds described herein are formed from bases that form non-toxic salts. Illustrative, albeit nonlimiting, examples include the arginine, benzathine, calcium, choline, diethylamine, diolamine, glycine, lysine, magnesium, meglumine, olamine, potassium, sodium, tromethamine and zinc salts. Hemi-salts of acids and bases, such as hemisulphate and hemicalcium salts, also can be formed.

The compounds can be formulated as pharmaceutical compositions and administered to a mammalian host, such as a human patient, in a variety of forms adapted to the chosen route of administration. For example, the pharmaceutical composition can be formulated for and administered via intraosseous, intravenous, intraarterial, intraperitoneal, intracranial, intramuscular, oral, nasal, and/or subcutaneous routes. In at least one embodiment, a compound and/or composition can be administered directly (via injection, placement or otherwise) to a TBI site. In at least one embodiment, the compounds can be systemically administered in combination with a pharmaceutically acceptable vehicle, such as an inert diluent or an assimilable edible carrier. For oral therapeutic administration, the active compound can be combined with one or more excipients and used in the form of ingestible tablets, buccal tablets, troches, capsules, elixirs, suspensions, syrups, wafers, and the like. The percentage of the compositions and preparations can vary and may be between about 1 to about 99% weight of the active ingredient(s) and a binder, excipients, a disintegrating agent, a lubricant, and/or a sweetening agent (as are known in the art). The amount of active compound in such therapeutically useful compositions is such that an effective dosage level will be obtained.

The preparation of parenteral compounds/compositions under sterile conditions, for example, by lyophilization, can readily be accomplished using standard pharmaceutical techniques well-known to those skilled in the art. In at least one embodiment, the solubility of a compound used in the preparation of a parenteral composition can be increased by the use of appropriate formulation techniques, such as the incorporation of solubility-enhancing agents.

As previously noted, the compounds/compositions can also be administered via infusion or injection (e.g., using needle (including microneedle) injectors and/or needle-free injectors). Solutions of the active composition can be aqueous, optionally mixed with a nontoxic surfactant and/or contain carriers or excipients, such as salts, carbohydrates and buffering agents (preferably at a pH of from 3 to 9), but, for some applications, they may be more suitably formulated as a sterile non-aqueous solution or as a dried form to be used in conjunction with a suitable vehicle, such as sterile, pyrogen-free water or phosphate-buffered saline. For example, dispersions can be prepared in glycerol, liquid PEGs, triacetin, and mixtures thereof and in oils. Under ordinary conditions of storage and use, these preparations can further contain a preservative to prevent the growth of microorganisms.

The pharmaceutical dosage forms suitable for injection or infusion can include sterile aqueous solutions or dispersions or sterile powders comprising the active ingredients that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example and without limitation, water, ethanol, a polyol (e.g., glycerol, propylene glycol, liquid PEG(s), and the like), vegetable oils, nontoxic glyceryl esters, and/or suitable mixtures thereof. In at least one embodiment, the proper fluidity can be maintained by the formation of liposomes, by the maintenance of the required particle size in the case of dispersions or by the use of surfactants or nanoparticle delivery modalities. The action of microorganisms can be prevented by the addition of various antibacterial and antifungal agents, such as parabens, chlorobutanol, phenol, sorbic acid, thimerosal, and the like. In certain cases, it can be desirable to include one or more isotonic agents, such as sugars, buffers, or sodium chloride. Prolonged absorption of the injectable compositions can be brought about by the incorporation of agents formulated to delay absorption, for example, aluminum monostearate and gelatin.

Sterile injectable or infusible solutions can be prepared by incorporating the active compound and/or composition in the required amount of the appropriate solvent with one or more of the other ingredients set forth above, as required, followed by filter sterilization. In the case of sterile powders for the preparation of sterile injectable solutions, the preferred methods of preparations are vacuum drying and freeze-drying, which yield a powder of the active ingredient plus any additional desired ingredient present in the previously sterile-filtered solutions.

Additionally, or alternatively, adjuvants, such as antimicrobial agents, can be added to optimize the properties for a given use. The resultant liquid compositions can be applied from absorbent pads, used to impregnate bandages and/or other dressings, sprayed onto the targeted area using pump-type or aerosol sprayers, or simply applied directly to a desired area of the subject (e.g., the site of TBI injury to the head).

Thickeners, such as synthetic polymers, fatty acids, fatty acid salts and esters, fatty alcohols, modified celluloses or modified mineral materials can also be employed with liquid carriers to form spreadable pastes, gels, ointments, soaps, and the like for application directly to the skin of the subject.

As used herein, the term “therapeutically effective dose” or “therapeutically effective amount” means (unless specifically stated otherwise) a quantity of a compound which, when administered either one time or over the course of a treatment cycle affects the health, well-being or mortality of a subject (e.g., and without limitation, reduces seizure susceptibility of a brain, reduces neuronal excitability in a brain, preserves BBB permeability, impairs injury-induced neuronal hyperactivity 30 days or more (e.g., 2-8 years) post-TBI, and/or prevents CTE development (e.g., up to 2 years post-TBI)). In certain embodiments, a therapeutically effective amount can be prophylactic. In certain embodiments, a therapeutically effective dose is a dose that is well below toxic levels to any part of the body of the subject.

In some embodiments, the therapeutically effective amount of any compound or pharmaceutical composition provided herein is determined in accordance with methods known in the art (e.g., animal models, human data, and human data for compounds that exhibit similar pharmacological activities). Useful dosages of the compounds can be determined by comparing their in vitro activity and the in vivo activity in animal models. Methods of the extrapolation of effective dosages in mice and other animals to human subjects are known in the art. Indeed, the dosage of the compound can vary significantly depending on the condition of the host subject, the severity of the TBI being treated, the route of administration of the compound and tissue distribution, and the possibility of co-usage of other therapeutic treatments (for example, in conjunction with the administration of hardware implantation, stem cell therapy and/or ultrasound therapies and the like). Indeed, the methods can further comprise administering a second therapy for treatment of a TBI to the subject, the second therapy comprising stem cell therapy, hardware implantation, ultrasound therapy, or administering a therapeutically effective amount of a second compound (such compound comprising, for example, an adenosine A₃ receptor agonist, an anticonvulsant, a coma-inducing drug, or a diuretic. A variety of anticonvulsants are commonly known in the medical arts and can include, without limitation, ethosuximide, zarontin, felbamate, felatol, fenfluramine, brivaracetam, cannabidiol oral solution, carbamazepine, cenobamate, clobazam, clonazepam, diazepam nasal, diazepam rectal, divalproex sodium, and eslicarbazepine acetate. Coma-inducing drugs are also commonly known in the medical arts and can include, for example, propofol, pentobarbital and thiopental.

In some embodiments, the therapeutically effective amount of any compound or pharmaceutical composition provided herein is determined by taking into consideration, for example, the potency of the type of therapeutic agent(s) employed (e.g., M-channel opener), body weight, mode of administration (e.g., s.c., i.v., or i.p.), severity of TBI, number of TBIs suffered, the like, or any combination thereof. The amount of the composition required for use in treatment (e.g., the therapeutically or prophylactically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise.

In some embodiments, the therapeutically effective amount of any compound or pharmaceutical composition provided herein is from about 0.01 mg/kg up to about 10 mg/kg. For example, therapeutically effective amounts or doses can range from about 0.03 mg/kg of patient body weight to about 30.0 mg/kg of patient body weight, or from about 0.01 mg/kg of patient body weight to about 1.0 mg/kg of patient body weight, including but not limited to 0.01 mg/kg, 0.02 mg/kg, 0.03 mg/kg, 0.04 mg/kg, 0.05 mg/kg, 0.1 mg/kg, 0.2 mg/kg, 0.3 mg/kg, 0.4 mg/kg, 0.5 mg/kg, 1.0 mg/kg, 1.5 mg/kg, 2.0 mg/kg, 2.5 mg/kg, 3.0 mg/kg, 3.5 mg/kg, 4.0 mg/kg, 4.5 mg/kg, and 5.0 mg/kg, all of which are kilograms of patient body weight. Intravenous doses can be several orders of magnitude lower. In some embodiments, the compound the therapeutically effective amount of any compound or pharmaceutical composition provided herein is administered (e.g., subcutaneously) in a single dose, or in sequential doses.

The total therapeutically effective amount of the compound can be administered in single or divided doses and can, at the practitioner's discretion, fall outside of the typical range given herein. In some embodiments, a therapeutically effective amount of any compound or pharmaceutical composition provided herein is administered once following a TBI event. In some embodiments, a therapeutically effective amount of any compound or pharmaceutical composition provided herein is administered once weekly following a TBI event. In some embodiments, a therapeutically effective amount of any compound or pharmaceutical composition provided herein is administered twice weekly following a TBI event.

It will be understood by one of ordinary skill in the relevant arts that other suitable modifications and adaptations to the compositions and methods described herein are readily apparent from the description contained herein in view of information known to the ordinarily skilled artisan and can be made without departing from the scope of the disclosure or any embodiment thereof

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

In the present description, numerous specific details are set forth to provide a thorough understanding of the present disclosure. Particular examples can be implemented without some or all of these specific details and it is to be understood that this disclosure is not limited to particular biological systems or particular organs or tissues, which can, of course, vary but remain applicable in view of the data provided herein.

Further, will be understood that the disclosure is presented in this manner merely for explanatory purposes and the principles and embodiments described herein can be applied to compounds and/or composition components that have configurations other than as specifically described herein. Indeed, it is expressly contemplated that the components of the composition and compounds of the present disclosure can be tailored in furtherance of the desired application thereof.

When ranges are used herein for physical properties, such as molecular weight, or chemical properties, such as chemical formulae, all combinations and sub-combinations of ranges and specific embodiments therein are intended to be included.

Additionally, the term “about,” when referring to a number or a numerical value or range (including, for example, whole numbers, fractions, and percentages), means that the number or numerical range referred to is an approximation within experimental variability (or within statistical experimental error) and thus the numerical value or range can vary between 1% and 15% of the stated number or numerical range (e.g., +1-5% to 15% of the recited value) provided that one of ordinary skill in the art would consider equivalent to the recited value (e.g., having the same function or result). The term “comprising” (and related terms such as “comprise” or “comprises” or “having” or “including”) is not intended to exclude that in other certain embodiments, for example, an embodiment of any compound, composition of matter, composition, method, or process, or the like, described herein, may “consist of” or “consist essentially of” the described features. The term “substantially” can allow for a degree of variability in a value or range, for example, within 90%, within 95%, or within 99% of a stated value or of a stated limit of a range.

Where a method of therapy comprises administering more than one treatment, compound, or composition to a subject, it will be understood that the order, timing, number, concentration, and volume of the administration is limited only by the medical requirements and limitations of the treatment (i.e., two treatments can be administered to the subject, e.g., simultaneously, consecutively, sequentially, alternatively, or according to any other regimen).

Additionally, in describing representative embodiments, the disclosure may present a method and/or process as a particular sequence of steps. To the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps disclosed herein should not be construed as limitations on the claims. In addition, the claims directed to a method and/or process should not be limited to the performance of their steps in the order written, and one skilled in the art can readily appreciate that the sequences may be varied and still remain within the spirit and scope of the present disclosure.

### EXAMPLES

The following examples illustrate certain specific embodiments of the present disclosure and are not meant to limit the scope of the claimed invention in any way.

### Example 1

### The Controlled Closed Cortical Impact Blunt TBI Model

Adult 10-week-old C57BL/6J mice (Jackson Labs) were group-housed in a 12:12-hour light:dark cycle with food and water ad libitum. Male and female mice were used for all experiments performed, except for GFAP, Iba1 and Fluro-Jade B histology experiments and for PSVue794 and Evans Blues imaging. As the previous experiments displayed no gender-specific differences, the present studies were only performed with male animals to avoid estrus cycle-related variation.

Animals were divided randomly among cohorts so that each cage included animals from all treatments. Cohorts of animals included “Sham” animals that received all procedures except the controlled closed cortical impact (CCCI), “TBI” animals subjected to CCCI and intraperitoneal (i.p.) injection of vehicle only, and “TBI+RTG” animals were subjected to CCCI followed by retigabine (RTG) administration 30 minutes after injury. The experimenters were blinded to group allocation during data collection and analysis.

The CCCI model was used to generate reproducible moderate blunt brain injury. This model has the advantages of producing focal contusion in which the skull remains intact. CCCI can most closely simulate blunt-head traumas resulting from falls, physical violence, or motor vehicle accidents and particularly in rodent models often produces spontaneous seizures within 24 hours.

The present investigations were restricted to moderate CCCI (without skull fracture or obvious hematomas) that can be associated with comorbidities that are possible to prevent. This model successfully reproduces the deleterious effects of TBI observed in the clinic, such as seizures, motor and cognitive deficits, brain edema, personality changes, mood disorders, and mitochondrial dysfunction.

A single trauma per mouse was employed. CCCI was delivered by a pneumatic impact device (Leica Biosystems) following a protocol previously published. A cylindrical probe of 5 mm diameter was used to deliver a calibrated impact to the skull, 1 mm depth over the right parietal cortex and a small portion of the caudal end of the right frontal cortex, at a velocity of 4.5 m/s to produce a moderate TBI. Sham mice underwent the same procedures of anesthesia and surgery, but without any impact.

Thirty minutes after CCCI, animals from the TBI+RTG group were injected i.p. with 1.2 mg/kg RTG (AdooQ Bioscience). Animals from the TBI group received i.p. injection of vehicle solution (0.5% (w/v) methylcellulose diluted in sterile 0.9% (w/v) saline). The dose used was the established maximally efficacious dose of RTG that was known to not be overly sedative to the point of reduction of requisite cerebral function (unaided respiration, swallowing, etc.). For example, such dosages cause much less sedation than the ordinary and common use of prescription benzodiazepines and “Z” class non-benzodiazepine sleep aids.

To quantify seizure frequency and determine seizure susceptibility, mice of each cohort were implanted with electroencephalogram (EEG) electrodes 24 hours after CCCI or Sham. A head mount was attached to a reference and three lead screws, 0.10″ and 0.12″, connected to a preamplifier (Gain 25X) (Pinnacle Technologies). The three leads were inserted over the right frontal and right and left parietal cortical areas, and the reference electrode was inserted over the left frontal cortical area. The head mount and electrodes were further secured to the skull with dental cement (Lang Dental).

Computer-assisted cortical EEGs was recorded and reviewed for the presence of seizures and epileptiform spikes. The Stellate Harmonie acquisition hardware and software was used to collect coincident video images of the animals with EEG activity and had several automated seizure detection algorithms. Continuous video/EEG monitoring was performed for three consecutive days, starting 24 hours after injury. Detected events were visually confirmed by blinded experimenters. All video/EEG recording data obtained are presented as the percentage of animals that displayed seizures or epileptiform spike activity. No animals in the Sham (control) group displayed any seizure or abnormal EEG activity.

To assay post-TBI seizure activity and assess for altered seizure susceptibility, mice from TBI and TBI+RTG cohorts were injected i.p. on the fifth day after CCCI, with three subthreshold doses of pilocarpine (MP Biomedicals, 75 mg/kg per dose), separated by 30-minute intervals. Pilocarpine is a cholinomimetic drug widely used as a chemo-convulsant in animal models to induce status epilepticus. To minimize pilocarpine-induced peripheral cholinergic effects and mortality, animals were pretreated with scopolamine methyl nitrate (i.p. 1 mg/kg; MP Biomedicals) 30 minutes prior to pilocarpine injection. Both drugs were diluted in sterile 0.9% (w/v) saline. Immediately after pilocarpine injection, cortical activity was monitored for 24 hours by video/EEG recording.

To quantify seizure frequency and determine seizure susceptibility, mice of each cohort were implanted with EEG electrodes 24 hours after CCCI or Sham. A head mount was attached to a reference and three lead screws, 0.10″ and 0.12″, connected to a preamplifier (Gain 25X) (Pinnacle Technologies). The three leads were inserted over the right frontal and right and left parietal cortical areas, and the reference electrode was inserted over the left frontal cortical area. The head mount and electrodes were further secured to the skull with dental cement (Lang Dental). FIG. 4C shows the schematic placement of the epidural screw electrodes use for EEG recording (left) and an image of a subject mouse fastened with an electrode head mount and preamplifier (right).

Computer-assisted cortical EEGs was recorded and reviewed for the presence of seizures and epileptiform spikes. The Stellate Harmonie acquisition hardware and software was used to collect coincident video images of the animals with EEG activity and had several automated seizure detection algorithms. Continuous video/EEG monitoring was performed for three consecutive days, starting 24 hours after injury. Detected events were visually confirmed by blinded experimenters. All video/EEG recording data obtained are presented as the percentage of animals that displayed seizures or epileptiform spike activity. No animals in the Sham (control) group displayed any seizure or abnormal EEG activity.

To assay post-TBI seizure activity, mice from TBI and TBI+RTG cohorts were injected i.p. on the 5^{th} day after CCCI, with three subthreshold doses of pilocarpine (MP Biomedicals, 75 mg/kg per dose), separated by 30-minute intervals. Pilocarpine is a cholinomimetic drug widely used as a chemo-convulsant in animal models to induce status epilepticus. To minimize pilocarpine-induced peripheral cholinergic effects and mortality, animals were pretreated with scopolamine methyl nitrate (i.p. 1 mg/kg; MP Biomedicals) 30 minutes prior to pilocarpine injection. Both drugs were diluted in sterile 0.9% (w/v) saline. Immediately after pilocarpine injection, cortical activity was monitored for 24 hours by video/EEG recording.

FIGS. 4A and 4B display the results. Whereas five of fifteen animals of the TBI group displayed post-traumatic seizures (PTS) manifested by both tonic-clonic behavior and abnormal increase in EEG amplitude and/or frequency of cortical signals, no animal treated with RTG post-TBI displayed seizures (χ(1)=4.16,p<0.05) (FIG. 4A). Regarding seizure susceptibility, in the RTG cohort of animals (TBI+RTG), FIG. 4B shows a significant reduction of pilocarpine-induced abnormal spike activity (χ(1)=3.96, p<0.05).

### Example 2

### Lactate/Pyruvate Quantification to Assay Cellular Energy Depletion

The ratio of lactate/pyruvate is a common measure of brain cell energy availability after injury. Accordingly, lactate and pyruvate were quantified in cortical samples collected 24 hours after CCCI from an independent cohort of mice used only this study, using the Lactate Colorimetric (BioVision) and Pyruvate Assay Kits (Elton Bioscience) according to manufacturer's protocols. For both, samples were tested in duplicates and average absorbances measured.

Mouse brains were bathed in ice-cold Ringer's solution and maintained over ice during dissection of cortical and cerebellar samples. Ipsilateral samples collected were from tissue of 2 mm×2 mm squares dissected around the impact area. The samples contained all cortical layers; hence, the 2 mm³ cube-shaped volume sample was level with the cortex in the dissected area. The contralateral cortical samples were collected in the corresponding area to that in the ipsilateral hemisphere.

For the Sham cohort, similar samples were collected from both hemispheres between the rostral and middle branches of the superior sagittal sinus, visible by eye, to where the TBI model was always localized. The cerebellum was also isolated and divided into contralateral and ipsilateral hemispheres. Since the right hemisphere was always the side to receive the CCCI, “ipsilateral” measurement was always from the right hemisphere. All samples were homogenized immediately after collection with lactate assay buffer (BioVision) and centrifuged at 4° C., 2,000 g for 10 minutes. Supernatants were collected and proteins removed by additional centrifugation at 4° C., 13,000 g for 10 minutes through 10 kD polyethersulfone spin filter columns (Thermo Fisher Scientific). Proteins were removed from the samples to prevent enzymatic degradation of lactate and/or pyruvate. Samples were frozen at −80° C. for future analysis.

Assay readings were executed in a Synergy HT plate reader (BioTek) at wavelengths of 450 (lactate) and 570 nm (pyruvate). Standard curves of 8 points from 9 to 200 μM were used to provide concentrations from linear regressions of the standard curves. Lactate/pyruvate ratios were obtained by dividing the lactate concentrations by the pyruvate concentrations independently for each sample.

A significant difference was observed between hemispheres and between Sham, TBI, and TBI+RTG cohorts in the cortex (the part of the brain in which most sensation, cognition and directed activity is located). In the cortex, only the TBI cohort displayed a significant increase in the lactate/pyruvate ratio in the ipsilateral versus the contralateral hemispheres (see FIG. 4D). Ipsilateral cortex samples from the TBI cohort also manifested higher lactate/pyruvate ratios, as compared to samples from Shams. In the TBI+RTG mice, the ipsilateral lactate/pyruvate ratio in the cortex was significantly lower as compared to the TBI cohort; indeed, the values of the TBI+RTG cohort were indistinguishable from those of the Sham cohort (see FIG. 4D). There were no TBI or RTG-induced effects observed in cerebellar samples of the mouse cohorts (see FIG. 4E). It is likely that the dramatic difference seem in the cortex are likely also observed in the hippocampus, the part of the brain most involved in memory storage.

### Example 3

### Cell Death and Blood-Brain Barrier Permeability

PSVue794, a near infra-red (NIR) fluorescent probe, was used to visualize the distribution of cell death throughout the brain. Through its bis(zinc²⁺-dipicolylamine) (Zn-DPA) targeting moiety, PSVue794 identifies apoptotic and necrotic cells by selectively binding to phosphatidylserines that are exposed to the outer surface of plasma membrane. PSVue794 (Molecular Targeting) was prepared the day of the experiment.

24 hours after CCCI, mice were injected through the lateral tail vein with PSVue794 (intravenous (i.v.) inj ection 3 mg/kg; Molecular Targeting) according to manufacturer's protocol. The probe was allowed to circulate for 48 hours prior to imaging to minimize non-targeted probe retention effect around peripheral tissues. At 72 hours post-CCCI, mice were euthanized and was performed to wash out the intravascular PSVue794. The entire brain was dissected from the skull, briefly kept in ice-cold phosphate buffered saline (PBS), and immediately placed into an IVIS Spectrum Imaging System (PerkinElmer). NIR fluorescence signals were detected ex vivo with the following settings: excitation filter, 710 nm; emission filter, 820 nm; exposure time, auto; binning, medium; f/stop, 2; field of view, A (as recommended in the IVIS operation guide).

To simultaneously assess blood brain barrier (BBB) permeability in a single animal, 72 hours after CCCI, mice were further injected with Evans Blue dye (i.v. 2.5 mg/kg; Sigma-Aldrich) through the lateral tail vein. The dye was allowed to circulate for 2 hours until mice were euthanized and transcardial perfusion was performed to wash out intravascular dye. Evans Blue has a high affinity to serum albumin and its fluorescence signal correlates with BBB disruption. The brain was dissected from the skull, briefly kept in ice-cold PBS, and immediately placed into an IVIS Spectrum Imaging System (PerkinElmer). The same cohort of animals went through both PSVue794 and Evans Blue imaging and thereafter ex vivo measurements were taken.

After PSVue794 imaging, brains were sliced with two sequential 2 mm sections (Sections A and B) around Bregma and further imaged to detected Evans Blue extravasation. Imaging was performed on both faces (rostral and caudal) of each section. Evans Blue fluorescence signals were detected ex vivo with the following settings: excitation filter, 640 nm; emission filter, 680 nm; exposure time, auto; binning, medium; f/stop, 2; field of view, A (as recommended in the IVIS operation guide).

Images obtained were analyzed using Living Image Software 4.5.5 (Caliper Life Sciences).

The values obtained with both probes scaled linearly with emission intensity and care was taken not to saturate the camera. Subjects were masked by adaptive fluorescence background subtraction to discard instrument background as explained in the Caliper Life Sciences Reference Guide. Graphical representations of the results obtained show Radiant efficiency (Emission Light (photons/sec/cm²/steradian)/Excitation Light (μW/cm²)) and values were divided by 106 (for PSVue794) and 107 (for Evans Blue) for normalization of data. Regions of interest (ROIs) were drawn around the peri-injured site of each sample and fluorescence values recorded. When Evans Blue and PSVue794 fluorescent signals were imaged independently but in the same animals, levels of BBB leakage and cell death were correlated to each other by means of fit to linear regression function.

FIG. 5A shows representative ex vivo images of NIR fluorescent dye PSVue794 probed brains, with the probe localized to the peri-injured area (with radiant efficiency illustrated by pseudocolor scale ranging from black (least intense) to red (most intense) and quantified in FIG. 5B. Cell death was observed three days after CCCI, which was significantly reduced by RTG administration (see FIG. 5B).

FIGS. 5C and 5D display data from the BBB disruption studies. As noted above, Evans Blue signals were obtained from four faces of the brain. The three groups of animals displayed significantly different levels of BBB permeability in all four areas (1-ANOVA—A rostral: F_{2,15}=6.4; A caudal: F_{2,15}=10.7; B rostral: F_{2,15}=23.2; B caudal: F_{2,15}=12.1). TBI mice manifested a significant increase in BBB permeability as compared to Shams in all four areas (see FIG. 5C), and TBI+RTG mice displayed a significant increase in BBB permeability in sections A rostral and B caudal, but had levels of BBB permeability similar to the Sham cohort in A caudal sections. TBI+RTG animals displayed significant decreased BBB breakdown vs. untreated TBI mice in sections A and B rostral. A linear correlation was also observed between cell death profile and BBB permeability in sections A caudal, B rostral, and B caudal.

### Example 4

### Immunostaining and Immunoblotting Measurements of the Mal-Adaptive Inflammatory Response

To assess immunological/inflammatory responses, expression of various biomarkers was quantified via immunoblotting of brain samples from both hemispheres six days after injury.

Brain slices previously processed six days after CCCI were used for immunohistochemistry. Slices stored at −80° C. on slides were brought to room temperature (RT) and dried overnight. The following day, they were rehydrated with PBS and immunoreactivity enhanced via heat-induced antigen retrieval using DIVA Decloaker (Biocare Medical). Slides were then washed with PBS twice for 5 minutes and dipped into 0.25% (v/v) Triton X-100 (Sigma-Aldrich) for another 10 minutes. Following two more PBS washes, sections were blocked with 5% (v/v) Bovine Serum Albumin (Sigma) for 1 hour at RT, excess blocking solution removed, and sections incubated with primary antibodies (1:1000 GFAP, Abcam ab53554; 1:500 Ibal, Wako 019-19741) at 4° C. for 20 hours. The following day, sections were washed with PBS 5× for 15 minutes/each and incubated in secondary antibodies (1:200 Thermo Fisher donkey anti-Goat Alexa Fluor 568; 1:200 Thermo Fisher, Chicken anti-Rabbit Alexa Fluor 488) for 1 hour at RT. They were then washed 3× for 5 minutes/each, and further incubated in DAPI (Sigma) for 10 minutes. After a brief wash in ddH₂O, slides were mounted with Vectashield antifade mounting medium (Vector Laboratories) and cover-slipped. All images were taken at 20× magnification (0.8 N.A.) using a Zeiss LSM710 confocal microscope. Fluorescence settings and parameters were held constant during each measurement.

For immunoblots, entire left and right hemisphere samples were collected six days post-CCCI and homogenized separately for quantification of immunoblots. Samples were homogenized using RIPA buffer (Thermo Fisher Scientific) and an Ultra EZgrind tissue homogenizer (Denville Scientific) at the lowest speed setting (5000 RPM). Immunoblot band intensities were normalized via the housekeeping proteins (3-actin or glyceraldehyde 3-phosphate dehydrogenase (GAPDH). Primary antibodies used were: Iba1 (1:400, Wako 016-20001), CD40L (1:500, Abcam ab52750), RIP-1 (1:1000, BD Biosciences 610459), GAPDH (1:25,000, Abcam ab125247), β-actin (1:20,000, Sigma A5316). Secondary independent normalization was also performed using Ponceau S (Sigma) staining and bands semi-quantified in the range of 37-100 kD. Results were standardized to ipsilateral averages from animals in the Sham cohort.

70 confocal images of each brain slice were taken by a Zeiss LSM710 confocal microscope under a 20× objective lens (Numerical Aperture=0.8). GFAP and Iba1 corrected total cell fluorescence (CTCF) values and the mean fluorescence intensities were quantified using ImageJ software.

Specifically, for CTCF, a single plane confocal image of the cerebral cortex was taken for both GFAP and Iba1 analysis, from each brain slice. The confocal plane used was selected based on the absence of holes or any other artifact. Each image underwent background subtraction. To identify cell bodies in the image (the ROIs), the desired binary images were generated by using the threshold option set at 5-15% for GFAP and 4-6% for Iba1 images. The following parameters were utilized to detect the ROIs automatically using the ImageJ function “analyze particles,” with size settings for GFAP and Iba1 at 40-8000 and 10-8000, respectively, and circularity of both GFAP and Iba1 at 0.0-10.0. With these parameters, ImageJ automatically detected cell bodies and defined them as ROIs. The mean gray value, integrated density, and ROI area were obtained along with background readings.

CTCF was calculated by subtracting from the integrated density of each cell the area of the ROI multiplied by the mean fluorescence of background readings. As for the mean fluorescence intensity analysis, the stacked images at 1 μm intervals were taken from each brain section. A customized ImageJ plugin from the Indiana Center for Biological Microscopy (ICBM) was employed to mask the images. The mean fluorescence intensity of the cortical region was calculated using automatic detection of positive pixels by the ICMB plugin. The results obtained for mean fluorescence intensity of the image and CTCF were averaged for each animal. Each average was used independently for statistical analysis and are represented as dots in the corresponding dot plot graph.

As shown in FIG. 6A (with (3-actin used as an internal control), significant differences were observed between samples from the two hemispheres, as well as among the different groups. The variation between groups was dependent on hemispheres, and the variation between hemispheres was dependent on cohort. A significant difference was seen between the ipsilateral vs. the contralateral side of animals in the TBI cohort, and between the ipsilateral side of animals in the TBI cohort vs. the ipsilateral side of Shams. There was also a significant reduction of Iba1 levels from ipsilateral samples from the TBI+RTG cohort vs. the TBI cohort.

To complement these results, the cortex slices immunostained for Iba1 expression exhibited a significant difference of mean total fluorescence between the three cohorts (see FIG. 6B, with the bars summarizing semi-quantification of immunoblots Iba1 in ipsilateral and contralateral hemispheres in each cohort (top), and the immunoblot bands for each hemisphere from each mouse group (below)), and between the two hemispheres within a cohort. Additionally, the variations between hemispheres and between groups were correlated (see FIGS. 6C and 6D). The ipsilateral cortex of TBI animals displayed greater staining for Iba1 than the ipsilateral side of the Sham and TBI+RTG groups, with greater Iba1 staining indicative of a greater immunological/inflammatory response in the tissue. Only the TBI group displayed a significant increase of staining in animals on the contralateral side of the TBI group (as compared to the Sham group). Further, significant differences in CTCF values were displayed between samples from the three cohorts (see FIG. 6E).

For GFAP expression (to assess astrogliosis), significant differences were observed across the three cohorts and the two cortical hemispheres (see FIGS. 6F-6H). There was also a significant interaction between these two factors, supporting that those changes in one parameter (group or cortical side) were related to the changes observed in the other. GFAP levels of TBI ipsilateral samples were significantly higher than the levels of GFAP in ipsilateral Sham samples and from TBI contralateral samples. TBI+RTG ipsilateral samples displayed lower GFAP levels than TBI ipsilateral samples, but higher levels as compared to Shams. Both TBI and TBI+RTG groups of mice displayed increased levels of GFAP in the contralateral hemisphere as compared to those of Shams. In addition, CTCF (see FIG. 61) was significantly different between samples from the cohorts.

The same brain samples as in FIG. 6A were used for semi-quantification of CD40L via immunoblotting (see FIG. 7A). A significant hemisphere-dependent effect was observed. For TBI animals, there was a robust increase in CD40L expression on the ipsilateral versus contralateral sides. However, in the TBI+RTG cohort, this effect was not observed. Interestingly, levels of CD40L were directly related to those of Iba1, as determined by fits of linear regression (not shown), supporting that TBI-induced increase in CD40L and increased microglia/macrophage activation/migration are correlated. However, CD40L was observed to be more localized to the site of impact, as no rise in CD40L levels was evidenced contralateral to the impacted hemisphere.

FIG. 7B shows the data related to RIP-1 immunoblots. A significant difference was identified in RIP-1 expression between cohorts. In TBI animals, RIP-1 expression increased between the ipsilateral, versus the contralateral, hemispheres. However, for the TBI+RTG group, no such difference was observed.

TBI also increased RIP-1 levels in the ipsilateral side of the tBI cohort as compared to the Shams cohort. Additionally, RIP-1 levels correlated with those of Iba1 or CD40L levels (data not shown).

### EXAMPLE 5

### Fluoro Jade B Measurement of Neuronal Cell Death

Flouro Jade B (FJB) staining was performed to assay neuronal cell death histologically and detect the degree of neurodegeneration six days after CCCI. Staining was performed as previously described.

Six days after CCCI, mice were euthanized via transcardial perfusion with PBS followed by 4% (w/v) paraformaldehyde (PFA). Brains were removed, post-fixed overnight in 4% PFA (Electron Microscopy Sciences), transferred into 30% (w/v) sucrose in PBS and kept at 4° C. for 3 days. Brains were then embedded into Peel-A-Way Disposable Histology Molds (TedPella) filled with Fisher Healthcare Tissue-Plus O.C.T. compound (Fisher Scientific), rapidly frozen with 2-methylbutane immersed in liquid N² and stored at −80° C. until cryosectioning.

30 μM thick coronal sections within the parietal cortex were prepared using a HM 505E Cryostat (Microm International), mounted on Superfrost Plus microscope slides (Fisher Scientific) previously coated with gelatin (Sigma), and dried overnight. Slides were stored at −80° C. until used. On the day of the experiment, all solutions were freshly prepared. Slides were kept in KPBS for 2 minutes and followed by dehydration steps in 50%-70%-100% ethanol for 2 minutes/each. Slides were rehydrated by repeating those steps in 70%-50% ethanol, KPBS 2 minutes each and incubated in potassium permanganate 0.06% for 5 minutes. After gentle rinsing in double distilled water (ddH₂O), slides were further incubated in Fluoro Jade B (Sigma Aldrich) solution for 10 minutes and rinsed in ddH₂O 3× for 1 minute/each. Stained slides were then dried, cleared in Xylene 3× each 2 minutes, and cover-slipped with DPX.

Ipsilateral cortical images (fluorescence images of FJB and counterstained DAPI) were taken by a Nikon Eclipse Ti with camera coolSNAP HQ² (confocal) microscope using a 20× objective (N.A. 0.75). Fluorescence excitation-emission for Fluoro Jade and DAPI were 470/40-500 and 350/50-460, respectively.

Cell count was obtained by nonbiased automatic quantification with ImageJ/FIJI (NIH, USA) particle analysis. All settings and parameters were held constant. After background subtraction and image threshold (1.05%), FIB positive cells were detected using the “analyze particles” (size: 20-100, circularity: 0.00-1.00) function of ImageJ. These parameters determined the pixel size and circularity of the acceptable ROIs and were used to identify cell bodies automatically.

No specific staining was observed in Sham samples and only weak staining was observed in in subcortical regions of TBI animals. TBI ipsilateral cortical slices, however, displayed significantly higher FJB staining as compared to TBI+RTG cortical slices (see FIG. 6J and 6K).

### EXAMPLE 6

### Tests Using Repetitive Blast TBI versus Single Blunt TBI

Adult 12-week-old male C57BL/6J mice (Jackson Labs, Bar Harbor, Me.) were group-housed with food and water ad libitum in 12:12 hour light: dark cycle. For c-Fos expression experiments, transgenic c-Fos TRAP mice (Fostm2.1(icre/ERT2)Luo/J) were crossed with mice that express the fluorescent protein tdTomato within a Cre-Lox recombination system (B6;12956-Gt(ROSA)26Sortm14(CAGtdTomato)Hze/J). Experimenters were blinded to group allocation during data collection and analysis.

A repetitive blast TBI (rbTBI) protocol was used that was similar to the protocol described in Example 1. The shock tube (Applied Research Associates) had and inner diameter of 17 inches and a about 36-inch end opening. It had a 24-inch driver with an 8-foot-long driven section and a 4-foot expansion cone with a 7.5-degree angle of expansion. Before reaching the mice, the air blast wave passed through a 0.016-inch-thick aluminum membrane of alloy AL-2200. Pencil probe gauges (PCB Piezotronics) were placed beside the mice to monitor the pressure of the air-driven blast wave. The airwaves presented the typical Friedlander waveform typical of that experienced by armed forces personnel, with an average maximum peak incident overpressure of 14.6 psi±0.5 and a positive phase of 2.8±0.5 milliseconds. During the procedure, mice were anesthetized using ketamine (25-75 mg/kg) and dexmedetomidine (0.25 mg/kg). The animals were subjected to three directed head-on blast TBI with 24 hours intervals between each injury. On the first day of blast exposure, animals were given a subcutaneous injection of buprenorphine SR (1.2 mg/kg) in the interscapular region.

Sham animals were anesthetized and placed in the shock tube but were not subjected to the rbTBI. After each blast exposure, mice were given one subcutaneous injection of atipamezole (1 mg/kg) to reverse the anesthesia and placed into a recovery chamber with controlled temperature. On the following days, mice were monitored for signs of pain and distress.

The mouse blunt TBI model used was the CCCI protocol described in Example 1. The CCCI model was used to generate reproducible mild/moderate brain injury. CCCI was delivered by a pneumatic impact device (Leica Biosystems). A cylindrical probe of 5 mm diameter was used to deliver a calibrated impact to the skull, 1 mm depth over the right parietal cortex and a small portion of the caudal end of the right frontal cortex, at a velocity of 4.5 m/s, to produce a moderate TBI. Sham mice underwent the same procedures of anesthesia and surgery but without any impact.

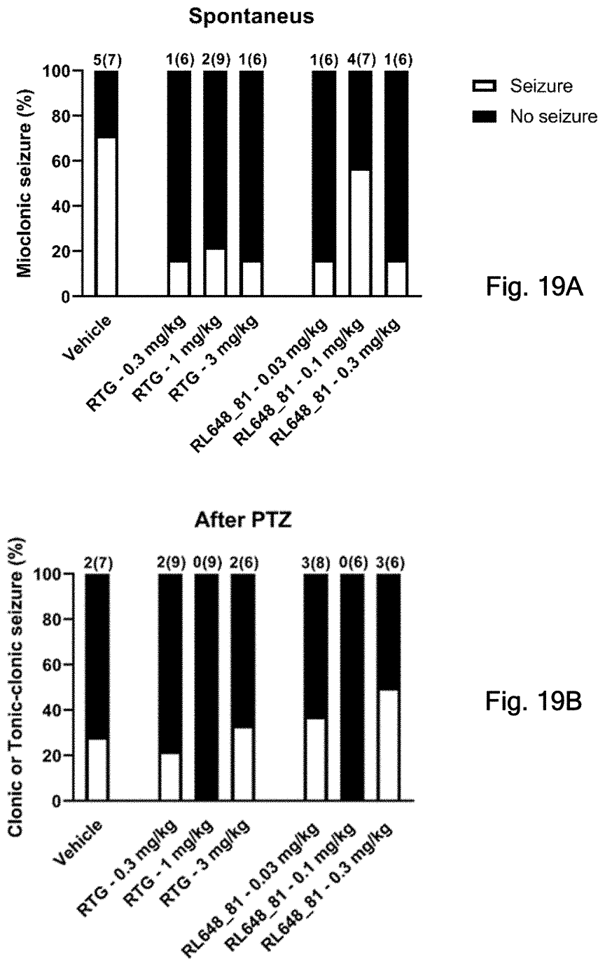

RTG dilution and administration were performed as previously described. RTG was diluted to a concentration of 330 μM in sterile 0.9% (w/v) saline solution with 0.5% (w/v) methylcellulose and 0.13% Dimethyl sulfoxide. RL648_81 (Sigma) was diluted in the same solution to a concentration of 26.93μM. 30 minutes after each blast TBI or blunt TBI exposure mice from the “TBI+RTG” group received one i.p. injection of RTG (1.2 mg/kg) and mice from “TBI+RL648_81” group received one i.p. injection of RL648_81 (0.1 mg/kg). Doses of RTG or RL648 81 varied only in the dose-effect experiment (see FIGS. 19A-19B). Time of injection varied only in the therapeutic window experiment (see FIG. 21). Mice from the “TBI” group were injected with the same volume of vehicle solution only.

To study the occurrence of seizures and the sleep-wake cycle architecture, EEG electrodes were implanted in the mice 24 hours after the last blast injury. Sham mice were also implanted with EEG electrodes, with video and EEG monitoring performed described in previous Examples. Prefabricated head mounts with screw electrodes (Pinnacle Technologies) were used. Four screws were placed inside the animal's head. A ground screw (left frontal lobe), a reference screw electrode (left parietal lobe), a right frontal lobe electrode, and a right parietal lobe electrode. Furthermore, the head mount also included a subcutaneous bipolar electrode in the medial parietal region in contact with the animal's skull. Starting one day after EEG surgery (2 days after the last blast injury) mice were video/EEG monitored for 72 hours. Afterward, mice were monitored for 48 hours every month for 8 months (starting one month after the last blast injury). For EEG recordings, the head mount was connected to a pre-amplifier (Gain 25X; Pinnacle Technologies) that was in turn connected to extracellular amplifiers of the Stellate Harmonie acquisition hardware. During all the recording sessions, mice were under 12:12-hour light: dark cycle with food and water ad libitum. During acquisition, the EEG signal was filtered with a 60 Hz notch noise filter. During seizure analysis, the EEG signal was filtered with a 0.1 to 45 Hz bandpass filter.

Seizures were identified visually by the investigator's analysis of the video/EEG records. The number of seizures, latency to the first seizure, seizure duration, and percentage of mice from each group that had seizures were quantified. Seizures displayed in the first 72 hours of record were considered PTS, whereas seizures that occurred one month or more after the last blast injury were considered as indications of post-traumatic epilepsy (PTE).

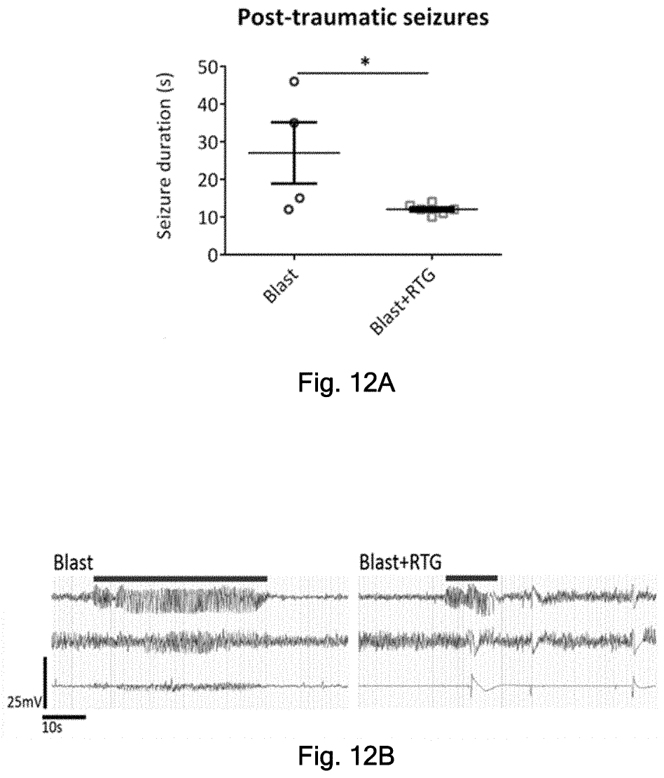

FIGS. 12A-12B illustrate seizure data of the mice who received rbTBI (mice subjected to three repetitive mild “blast” or “shockwave” TBIs). The rbTBI+RTG cohort that received the acute treatment 30 minutes following each blast exhibited significantly reduced duration of the PTS as compared to the rbTBI cohort that did not receive treatment with RTG (i.e., that received vehicle only) (see FIG. 12A). Furthermore, the EEG recordings shown in FIG. 12B support that the mice of the rbTBI+RTG cohort that were acutely treated post-TBI experienced not only seizures of shorter duration, but are also less severe (decrease in amplitude as compared to rbTBI group that did not receive RTG).

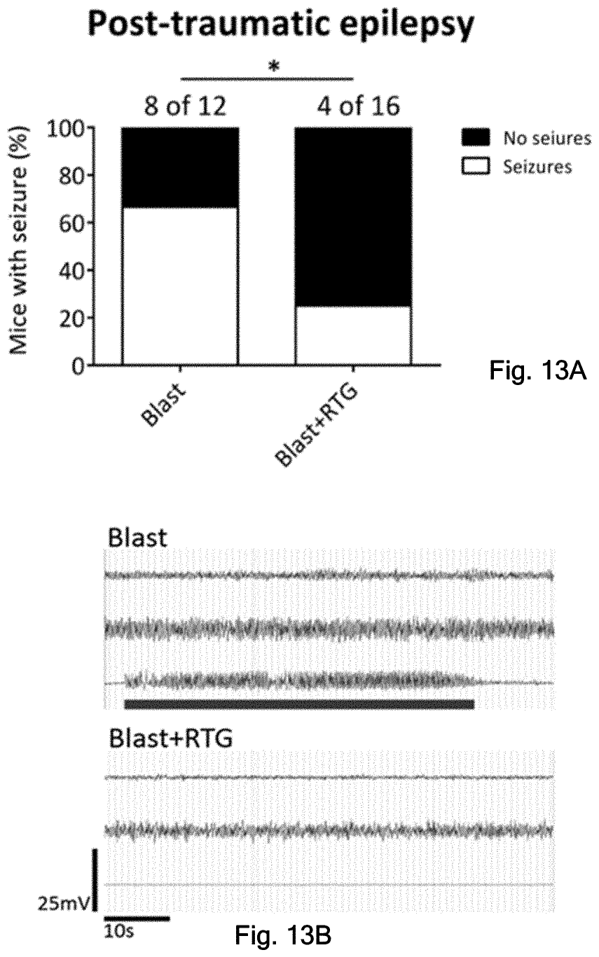

In addition to acute effects, treatment with RTG administered 30 minutes after each TBI significantly reduced the percentage of mice that developed PTE (see FIGS. 13A and 13B), with over 60% of mice in the Blast group experiencing seizures as compared to about 20% of mice in the rbTBI+RTG group experiencing seizures. Mice were video monitored from the first to the ninth month after the final rbTBI was administered (8 months total, 48 hours per month).

### Example 7

### Effects on the Sleep-Wake Cycle

To study the sleep-wake cycle, video/EEG records from the first 72 hour long record session (starting 2 days after the last injury) and the last 48 hour long record session (9 months after the last injury) were analyzed. To select one EEG channel from each animal/session for further analysis, we performed visual inspections of the raw local field potentials and their time-frequency decompositions (spectrograms) recorded from the three electrodes. EEG channels with signal saturation and background noise or artifacts were discarded. Then, the EEG channel with the highest percentage power at the 5-12 Hz frequency band was used to classify the sleep-wake cycle. Awake state (WK), slow wave sleep (SWS), rapid eye movement sleep REM), and transition/undetermined states (US) were classified based on two power spectral ratios, as described by Gervasoni et al., Global Forebrain Dynamics Predict Rat Behavioral States and Their Transitions, J Neurosci 24(49): 11137-11147 (2004). In brief, it first measures the relative power within fast (0.5-20/0.5-50 Hz) and slow (0.5-4.5/0.5-9 Hz) frequency bands and then generates a 2 dimensional (2D) state-space on which clusters are manually selected and associated with each state of the sleep-wake cycle.

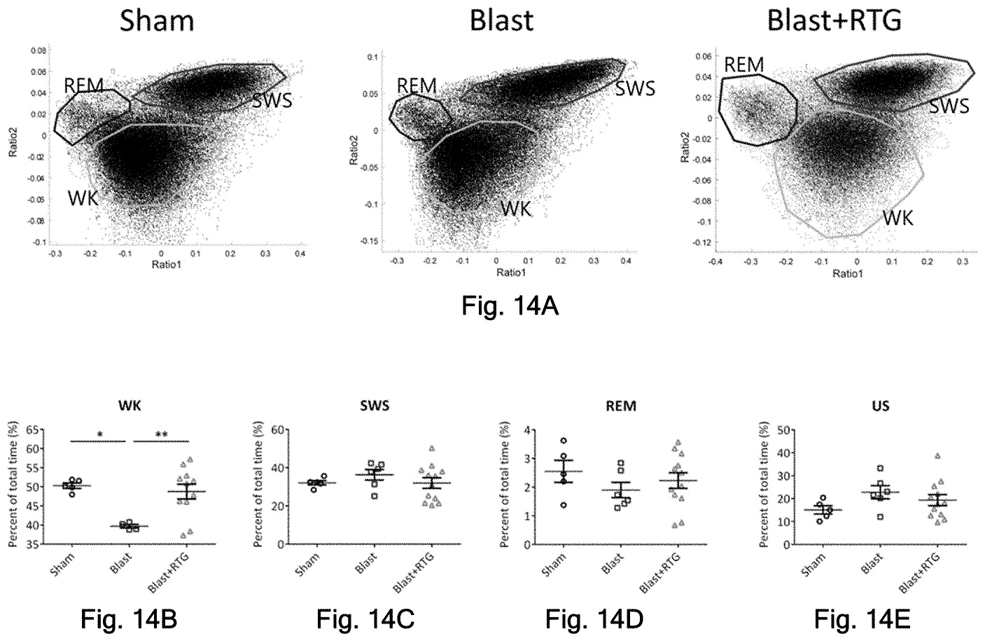

FIG. 14A-14E show data from the component analysis of the EEG recordings. Significant changes were identified between awake state (WK) in the first week post-rbTBI treatment, while no significant effect was observed in the time spent in slow wave sleep (SW), rapid eye movement sleep (REM), or transition/undetermined state (US). The rbTBI-RTG cohort experienced percentage of time in awake state roughly equivalent to that experienced by the Sham group, whereas the Blast cohort experienced at least a 10% reduction in awake state post-rbTBI.

### Example 8

### Analysis of Effect on the EEG Signal Power

Aging induces an increase in the power (amplitude) of “Gamma” cortical waves during slow wave sleep. This is a normal physiological process important for a healthy aging brain as compared to pathological. To assess if the power of the EEG signal was affected by TBI, gamma signals were assessed.

The power of the EEG signal in the different frequencies was calculated using the “pwelch” function of the computer program Matlab. In brief, the power of each frequency is decomposed in a distribution histogram. The powers of the EEG signal in the gamma (30-50 Hz) and the delta (2-9 Hz) frequencies were calculated as the average of the power in these range of frequencies. The average power obtained was then divided by the average power of the EEG signal in all the frequencies. Therefore, the power of the gamma or the delta frequencies is presented as a percentage of the total power of the EEG signal. The power of the EEG signals was calculated during the SWS state. The power analysis reflected the amplitude of a wave in a specific frequency.

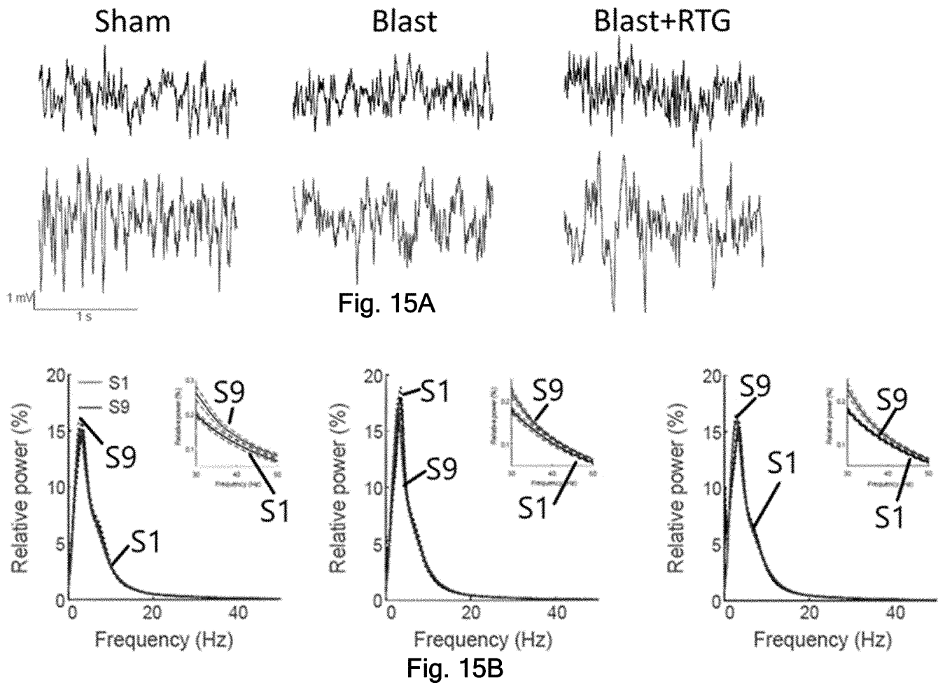

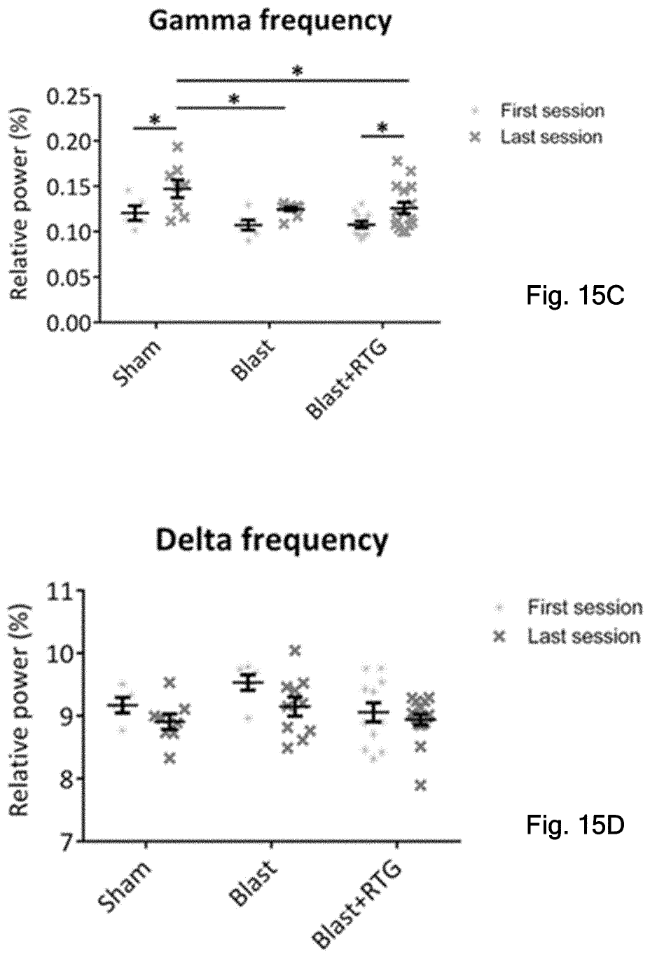

As shown in FIGS. 15A-15D, gamma cortical waves that increase with age in a normal subject were impaired by rbTBIs. However, acute post-TBI RTG treatment reduced this deleterious effect of the TBIs observed 9 months after the last blast TBI. As shown in FIG. 15D, the delta frequency was not affected by either blast TBIs or by administration of RTG.

### Example 9

### TBI-Induced Accumulation of TAR DNA-binding Protein 43, a marker of CTE

Overexpression of TAR DNA-binding protein 43 (TDP-43) in the cerebral cortex is a widely-accepted marker of Chronic Traumatic Encephalopathy (CTE), a type of cognitive impairment widely seen not only after blast TBI, but in contact sports and chronic domestic violence. Acute RTG treatment after multiple mild blast TBIs (rbTBIs).

Levels of TDP-43 were first analyzed by immunoblotting experiments performed as previously described. Cortical and hippocampal samples were collected when the mice were 26-28 months of age.

Complementary to the immunoblot experiments, immunohistochemistry staining of TDP-43 was performed as previously described. Left hemisphere samples were removed and fixed overnight in 4% paraformaldehyde (Electron Microscopy Sciences). Subsequently, samples were transferred into 30% (w/v) sucrose in PBS and kept for 3 days at 4° C. before they were frozen in Tissue-Plus O.C.T. Compound (Fisher Scientific) using 2-methylbutane immersed in dry ice. Samples were stored at −80° C. until cross-sectioning.

20 μm thick coronal sections of the prefrontal cortex (PFC) were mounted on Superfrost Plus microscope slides (Fisher Scientific) previously coated with gelatin (Sigma). Before staining, slices were blocked with 8% (v/v) donkey serum with triton-x (0.1% v/v) and Tween (0.1% v/v). The primary antibodies used were TDP-43 (1:200, Proteintech 12892-1-AP) and NeuN (1:200, Millipore MAB377). Slides were mounted using an antifade mounting medium with 4′,6-diamidino-2-phenylindole (DAPI) (Vector Laboratories) for nuclear staining. Images were taken in the PFC layer II at 40× magnification using an iXon ultra EMCCD Andor camera attached to a Nikon Eclipse TE2000-U microscope, with a fixed exposition time of 200 ms, in the 16-bit readout mode, with 2 MHz speed, and a pre-amplifier gain of 3. The mean and maximum fluorescence signal was obtained by automatic quantification with ImageJ (National Institutes of Health, USA). Regions of interest were determined automatically using the “analyze particles” function of ImageJ (size: 50-8000 pixel \hat{ }2; circularity: 0.00-1.00).

Samples used in the immunoblotting and the immunohistochemistry experiments were from the EEG-monitored mice and from the above independent cohort. Samples for immunoblotting analysis were from the left hemisphere, whereas the right hemisphere was used for immunohistochemistry. Samples for immunoblotting experiments were dissected in ice-cold Ringer's solution, snap-frozen in dry ice, and stored at −80° C. until analysis. Samples were homogenized in RIPA buffer (Thermo Scientific) using a tissue homogenizer (Ultra EZgrind—Denville Scientific) at 5,000 rpm. Proteins were separated on 20% agarose gels (Bio-Rad) and proteins transferred to polyvinylidene difluoride (PVDF) membrane (Bio-Rad). The primary antibodies used were TDP-43 (1:1000, Proteintech 12892-1-AP), pTDP-43 (1:1000, Cosmo Bio TIP-PTD-M01), and β-actin (1:20,000, Sigma A5316). Results were standardized to the average of the results from Sham mice.

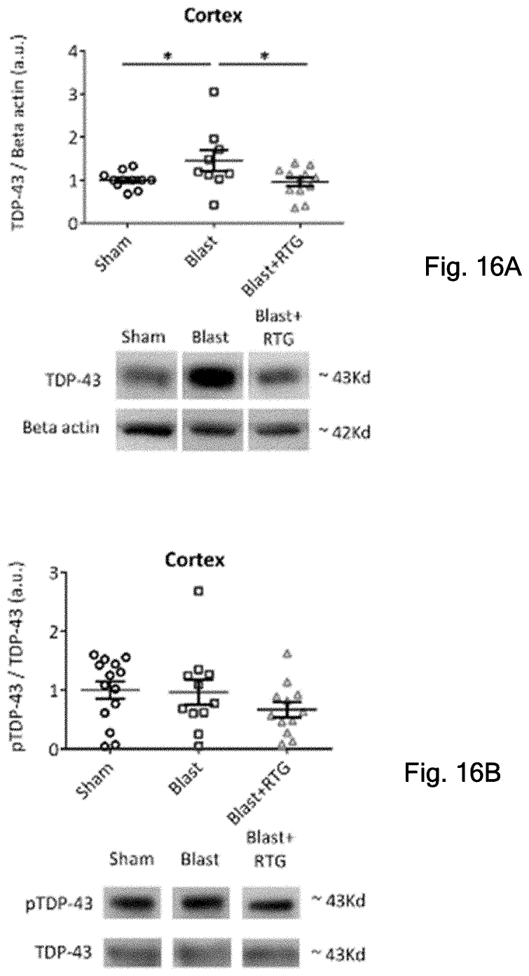

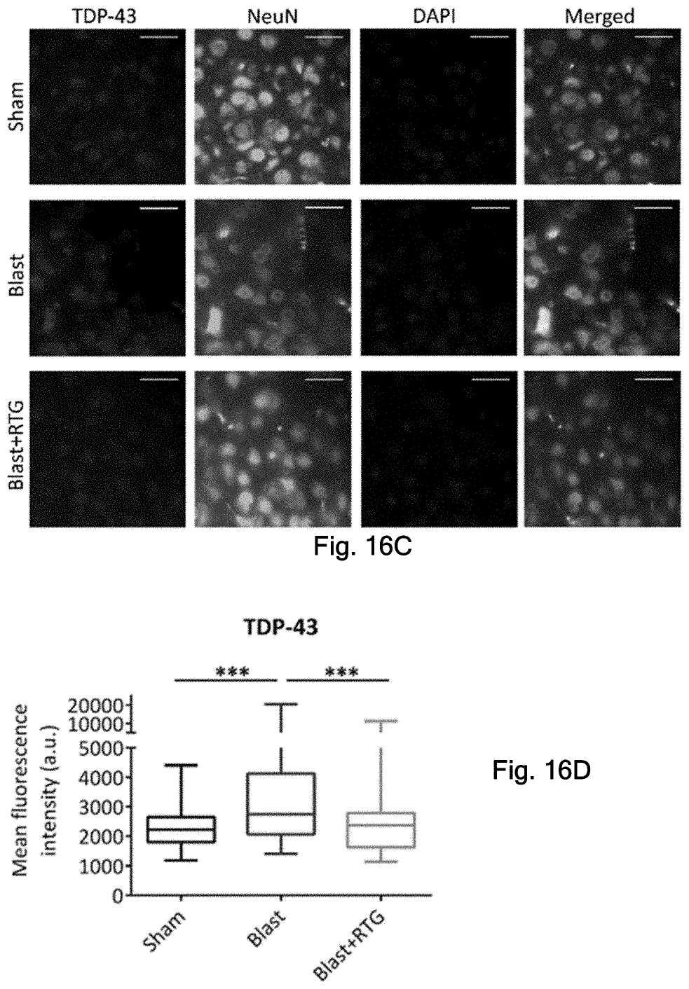

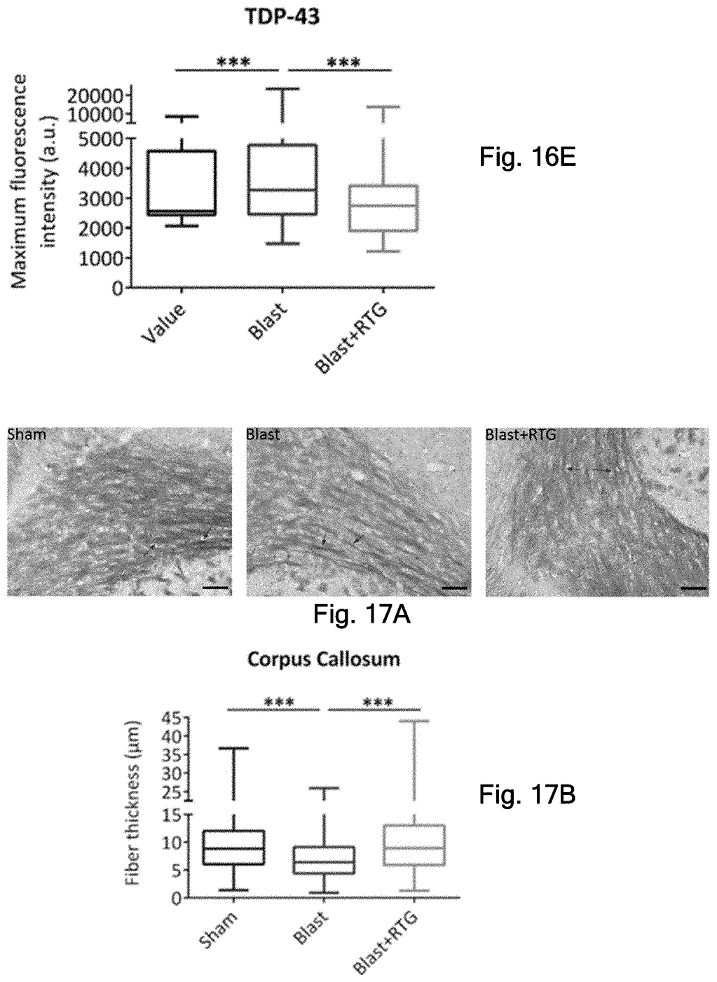

As shown in FIGS. 16A-16E, in each instance, the Blast-RTG treatment cohort expressed reduced TDP-43 as compared to the Blast cohort. FIG. 16A shows the summarized immunoblotting analysis of total TDP-43 expression, normalized to β-actin expression. Neither TBI, nor RTG treatment, changed the phosphorylation levels of TDP-43 (see FIG. 16B). This supports RTG treatment after multiple mild blast TBIs prevented long-term (2 years after TBIs), TBI-induced accumulation of TDP-43 in the cerebral cortex and presumably CTE development in those animals.

### Example 10

### Effects on Nerve Fiber Degradation

Nerve fiber degradation is another widely-accepted markers of CTE. As such, nerve fiber degradation was assessed in each of the study cohorts to determine the effect, if any, thereon by acute RTG treatment following rbTBI.

Tissues collected for immunohistochemistry staining of TDP-43 were also used for Luxol fast-blue staining. Twenty μm thick coronal sections of striatum and corpus callosum were stained to assay the integrity of nerve fibers. These brain areas were selected due to the high content of myelinated fibers. To stain the myelin sheets, slices were incubated in Luxol fast blue solution (Sigma) overnight at 60° C. Eosin Y stain (Ricca Chemical Company), and cresyl violet (0.1% w/v, Abcam) were used for counterstaining. Images were taken in an Olympus microscope (BX 60) with an Olympus camera (DP-71) and a 10× objective. Images were analyzed using ImageJ. In the corpus callosum, the diameter of the nerve fibers was measured. In the striatum the area of the fiber was determined.

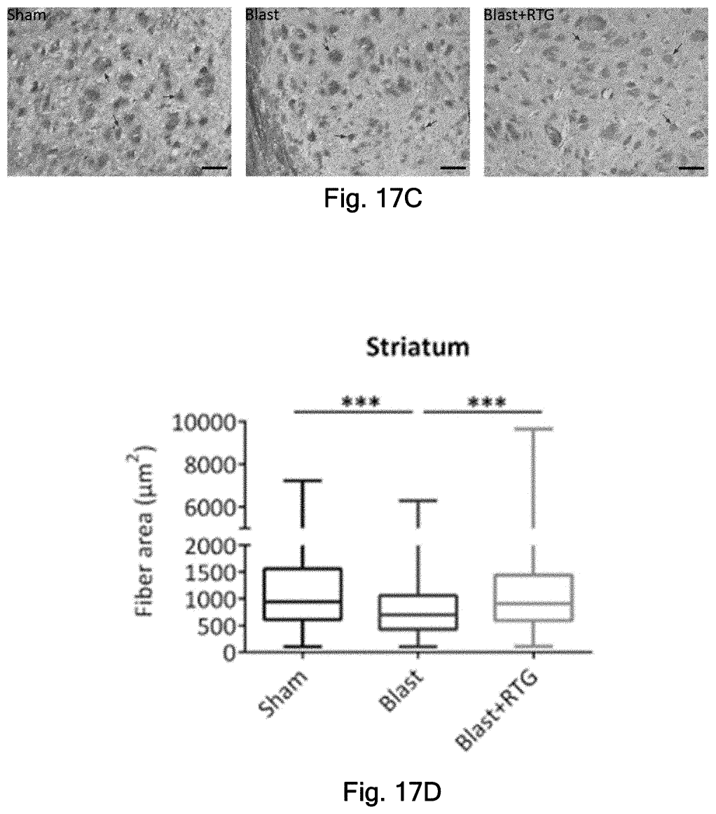

The resulting data shown in FIGS. 17A-17D supports that acute post-TBI treatment after rbTBI prevents the degradation of nerve fibers in two critical brain areas two years after multiple mild blast (shockwave) TBIs: the corpus callosum that connects the two hemispheres of the brain (FIGS. 17A-17B) and the striatum of the basal ganglia in the forebrain, which is a critical brain region responsible for motor function, reward circuits, cognition, action planning, and decision making (FIGS. 17C-17D). The myelin in FIGS. 17A and 17C is labeled with Luxol fast-blue staining.

### Example 11

### Effects on Neuronal Activity in Sub-Cortical Regions

Transgenic “FosTRAP” mice were used to study neuronal activity in sub-cortical regions following blunt TBI (the CCCI model as described above). These mice express the fluorescent protein, tdTomato, under the control of the neuronal promoter c-Fos, an “early intermediate gene” transcriptional factor that correlates well with heavy neuronal activity. Accordingly, whenever neurons express c-Fos, tdTomato is also expressed, allowing for an optical reporter of prior neuronal activity.

Because these transgenic mice express tdTopmato via the tamoxifen-inducible Cre/IoxP system, mice were injected (i.p.) with tamoxifen (75 mg/kg) 30 days after a single blunt TBI (i.e., CCCI), and samples were collected 5 days after injection.

Slices (200 μm) of each collected sample were infused with 4% (w/v) PFA for 10 minutes at RT, washed 5× in PBS and blocked with 8% (v/v) donkey serum (Sigma-Aldrich) diluted in PBS, 0.1% (v/v) Triton X-100 (Sigma-Aldrich), and 0.1% (v/v) Tween 20 (Thermo Fisher Scientific). After blocking for 2 hours at RT, followed by washing 5× in PBS, slides were mounted with Vectashield antifade mounting medium with nuclear staining DAPI (Vector Laboratories) and cover slipped. A Nikon Eclipse FN1 microscope with 10× and 40× Plan Fluor objectives was used for detection of fluorescence. Images were obtained at a fixed exposure time of 5 seconds (averaging 8 frames). Cell count was obtained by non-biased automatic quantification with ImageJ. The image threshold was set at 15-25% to facilitate ROI identification. ROIs were determined automatically using the “analyze particles” function of ImageJ, with size set to 200-8000 and circularity set to 0.00-1.00.

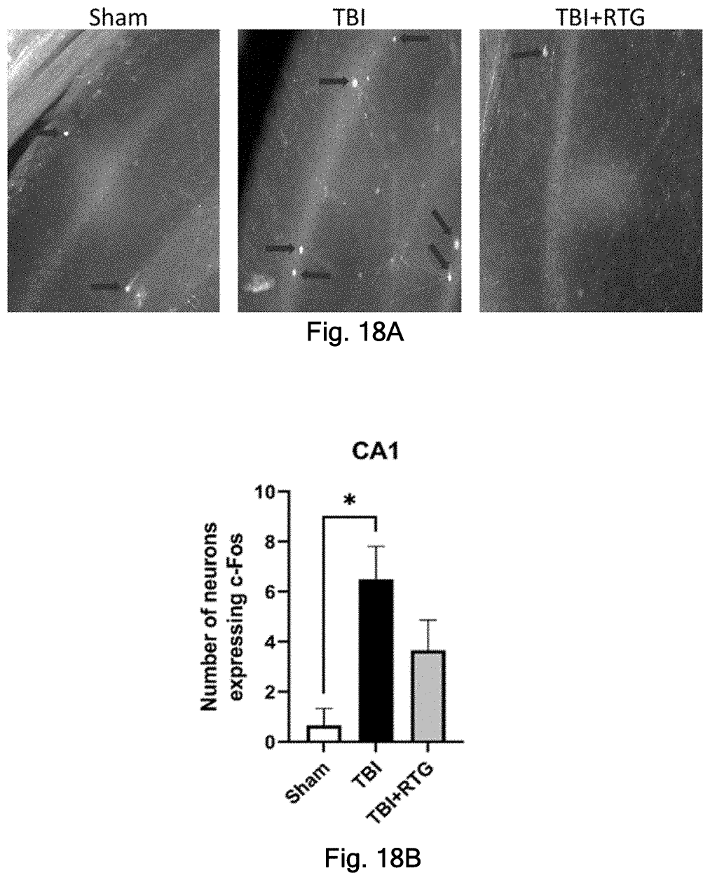

FIGS. 18A show exemplary images of hippocampal CA1 slices from all three cohorts (Sham, TBI, and TBI RTG), with the arrows indicating examples of neurons counted as c-Fos positive. FIG. 18B shows a graphical summary of the increased c-Fos expression that was blunted by acute RTG treatment, with the TBI-RTG cohort expressing significantly less c-Fos than the TBI cohort.

### EXAMPLE 12

### Dosage Optimization

Three doses of RTG (0.3 mg/kg, 1 mg/kg, and 3 mg/kg) were tested on mice following one blunt TBI utilizing the experimental protocols previously described in connection with the CCCI model to optimize dosages and efficacy. An additional KCNQ2/3-specific compound, RL648_81 was also administered in the same manner (0.03 mg/kg, 0.1 mg/kg, and 0.3 mg/kg). RTG andRL648_81 treatments were injected once 30 minutes post-TBI (i.p.). To monitor the occurrence of spontaneous myoclonic seizures after, the mice were video/EEG monitored from the second to the fourth day after the TBI (72 hours total).

As supported by FIG. 19A, all three dosages of RTG tested significantly reduced the occurrence of myoclonic seizures post-TBI. RL648_81 also measurably reduced the occurrence of TBI-induced myoclonic seizures, more significantly at the lowest (0.03 mg/kg) and the highest (0.03 mg/kg) doses.

To test dosage efficacy seizure susceptibility, dosages of RTG and RL648_81 were also tested on the cohorts (Sham, TBI and TBI+treatment (whether RTG or RL648_81)) when stimulated with pentylenetetrazol (PTZ), a chemo-convulsive agent. Specifically, PTZ was administered to each cohort at a normally sub-seizure threshold dose (35 mg/kg; s.c.) on the fifth day post-TBI in conjunction with the vehicle (TBI cohort), and in the treatment groups, in conjunction with RTG or RL648_81 (TBI-RTG identified as “RTG—[dosage]” and “RL648_81—[dosage]” in FIG. 19B, respectively). Stimulation with PTZ induced the occurrence of clonic and tonic-clonic seizures in TBI-subjected mice. RTG at 1 mg/kg and RL648_81 at 0.1 mg/kg completely blocked the occurrence of PTZ-induced seizures. Notably, these results support (at least in part) that at or about the same dose of RTG can be used to prevent both spontaneous seizures and decrease seizure susceptibility.

### Example 13

### Two-Photon In Vivo Imaging Showing Changes to BBB Permeability

In vivo two-photon confocal microscopy was used to study the BBB permeability 2 hours after one blunt TBI (CCCI model). The cranial window approach was used as it allows for the imaging of blood vessels and surrounding parenchyma in vivo without anesthetizing the mouse. A glass cranial window was placed in the animal's head replacing a small piece of the skull as described below. The cranial window surgery was divided into two days.

On the first surgery day, the mice were chemically anesthetized with i.p. injection of solution composed of saline (0.9% NaCl), ketamine 10% (v/v), and xylazine 1% (v/v). The animal temperature was monitored and a heating pad was used to maintain proper body temperature. During surgery mice anesthesia was monitored with test for rear foot reflexes, and the breathing rate monitored. A midline incision in the scalp was then made and the skin and periosteum retracted, exposing the skull. A stainless metal plate (8 mm wide and 3.3 cm long), with a hole (5 mm diameter) in the center of the plate, was attached to the exposed area of the skull using adhesive resin cement) (C&B-METABOND®). This concluded the first day of surgery. The mouse was then placed in a warmed cage until it was fully awake and returned to his home cage. The animal was monitored for one week while recovering.

For the second day of procedure, the animal was once more anesthetized with chemical anesthetic. Animals were again placed over a heating pad with body temperature and the anesthetic plane was monitored constantly. The resin cement located inside the hole in the metal plate was drilled using a round head micro drill steel burr of 0.5 mm. Once the resin in the hole was drilled and the skull exposed, a ˜1.7 mm² area of the skull was carefully removed with scalp incisions. The brain was exposed and immediately covered with a bubble of sterile-filtered saline solution. The dura matter was carefully removed, and a glass coverslip window was placed over the exposed brain. Finally, the window was fixed in place with orthodontic acrylic resin (Ortho-Jet™), after which the animal was placed in a recovery cage and monitored until fully awake and moving around its cage. The mice were left in his home cage to recover from surgery for a week before initiating habituation for two-photon imaging. For habituation to the imaging procedure, mice were placed under the microscope for 1 hour every other day, 3 times before experiments started.

Changes in BBB permeability were assayed in Sham, vehicle-treated (TBI), RTG-treated (TBI+RTG), and RL648_81-treated (TBI RL648_81) mice after i.v. injection of TRITC-dextran (150 kD; 133 mM; 150 μL). TRITC-dextran is a large molecular weight fluorescent molecule that is not permeable through an intact BBB. The molecule is labeled with the red fluorescent dye, TRITC, to permit optical observation of dextran permeability through the BBB. Mouse brains were imaged before and 2 hours after CCCI and TRITC-dextran was injected 30 minutes before imaging (1.5 hours post-blunt TBI). For each mouse, images were taken from six different areas of the primary visual cortex Layer 2/3 before and 2 hours after one CCCI. The two-photon laser was set to 800 nm excitation laser. TRITC-dextran positive area outside the blood vessels was measured for each image.

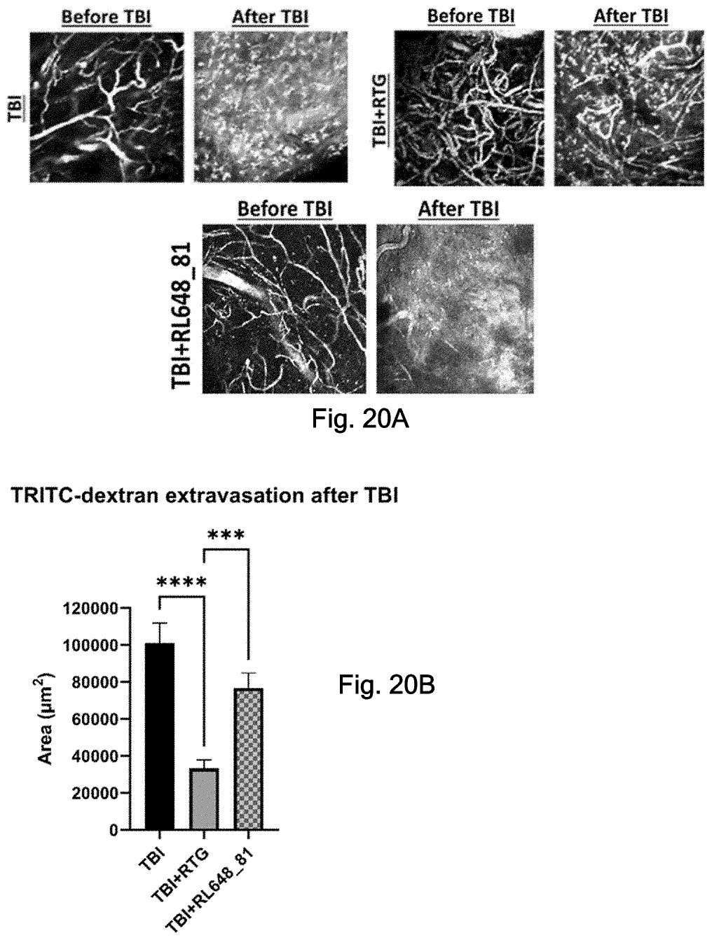

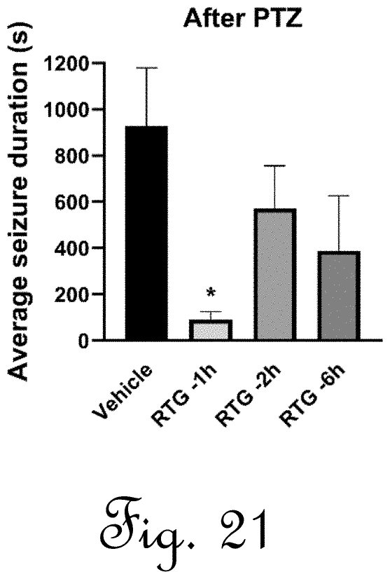

The TRITC-dextran in vivo 2-photon confocal imaging experiments revealed blunt TBI induced breakdown in BBB permeability (2 hours after TBI) in the TBI cohort; however, that breakdown was strongly occluded by acute RTG treatment (1 mg/kg) 30 minutes post-TBI such that BBB integrity was preserved. The occlusive effects were not observed from treatment with RL648_81 (0.1 mg/kg). FIG. 20A shows representative images of TRITC-dextran florescence restricted to the brain blood vessels before the blunt TBI and its extravasation into the parenchyma of the cortex 2 hours post-TBI, and FIG. 20B shows a graphical representation summarizing the data of FIG. 20A.

### Example 14

### Treatment Window Studies

In other examples, for the TBI-RTG cohort, RTG was injected 30 minutes post-TBI. Here, the effective window for treatment with RTG following one blunt TBI (CCCI model) was assessed. Mice were video-EEG monitored for 24 hours after stimulation with a normally sub-seizure threshold dose of PTZ (35 mg/kg; s.c.) administered on the fifth day post-TBI, which caused the mice to respond with prolonged seizures. Treatment with RTG (1 mg/kg; i.p.) was performed at 1-hour post-TBI, 2 hours post-TBI, and 6 hours post-TBI. A TBI cohort (vehicle only) was also assessed for control purposes.

As shown in FIG. 21, treatment with RTG at 1-hour post-TBI significantly reduced induced seizure duration. Lesser effects were observed with RTG treatment administered 2 hours and 6 hours post-TBI.

For all experiments described herein, percentages of mice in each group that displayed seizures were analyzed using the Pearson chi-square test. All other data were checked for normal distribution with the Shapiro-Wilk normality test and Q-Q plot analysis. If the data showed a non-normal distribution, outliers were excluded from the experiment as a normalization strategy. Outliers were defined as values that fall more than 1.5 times the interquartile range below the first quartile or above the third quartile. If this normalization strategy was not effective, outliers were returned to the dataset and the data considered to have a non-normal distribution. Non-normally distributed data were analyzed using the Kruskal-Wallis test followed by Mann-Whitney U (MWU) test with Bonferroni correction as post hoc analysis. The data are presented here using box and whisker plots in which minimum, first quartile, median, third quartile, and maximum values are displayed. For normally distributed data, ANOVA and Student-Newman-Keuls (SNK) post hoc tests were used. Normally distributed data are presented using scatter plot graphs with mean and SEM.

1. A method for treating brain injury or dysfunction resulting from at least one traumatic brain injury, the method comprising:
administering a therapeutically effective amount of a compound comprising an M-channel opener to a subject after the subject experiences a traumatic brain injury.

2. The method of claim 1, wherein the M-channel opener upregulates at least a KCNQ2 subunit, a KCNQ3 subunit, or both of an M-channel of a brain of the subject.

3. The method of claim 1, wherein the M-channel opener is retigabine, a derivative thereof, or a pharmaceutically acceptable salt thereof.

4. The method of claim 3, wherein the M-channel opener is RL648_81 or a pharmaceutically acceptable salt thereof.

5. The method of claim 1, further comprising reducing neuronal excitability in a brain of the subject via exposure to the M-channel opener.

6. The method of claim 1, further comprising reducing cellular energy demand in a brain of the subject via exposure to the M-channel opener.

7. The method for treating brain injury or dysfunction of claim 1, wherein administering the therapeutically effective amount of the compound prevents development of chronic traumatic encephalopathy (CTE) in a brain of the subject for at least two years following traumatic brain injury.

8. The method of claim 1, wherein administering is delivered intravenously, intramuscularly, subcutaneously, transdermally, orally, or nasally.

9. The method of claim 1, further comprising preserving permeability of a blood brain barrier of a brain of the subject via exposure to the M-channel opener.

10. The method of claim 1, wherein the at least one traumatic brain injury is selected from the group consisting of: blast injury, blunt trauma, Shaken Baby syndrome, concussion, and concussion syndrome.

11. The method of claim 1, wherein administering the therapeutically effective amount of the compound is performed within 1 hour of the subject experiencing the at least one traumatic brain injury event.

12. The method of claim 12, wherein administering the therapeutically effective amount of the compound is performed within 30 minutes of the subject experiencing the at least one traumatic brain injury event.

13. The method of claim 1, wherein the therapeutically effective amount of the compound is administered at between 0.3 mg/kg-3.0 mg/kg of body weight of the subject.

14. The method of claim 13, wherein the therapeutically effective amount of the compound is administered at 1.0 mg/kg of body weight of the subject.

15. The method of claim 1, further comprising administering a second therapy for treatment of the traumatic brain injury to the subject, the second therapy comprising stem cell therapy, hardware implantation, ultrasound therapy, or a therapeutically effective amount of a compound comprising an adenosine A3 receptor agonist, an anticonvulsant, a coma-inducing drug, or a diuretic.

16. A method of reducing a subject's susceptibility to experiencing a seizure or cognitive dysfunction following a traumatic brain injury, the method comprising:
administering a therapeutically effective amount of a compound comprising an M-channel opener to a subject who has experienced a traumatic brain injury,
wherein administration occurs within six hours after the traumatic brain injury.

17. The method of claim 16, wherein the subject is experiencing or at risk of experiencing metabolic exhaustion in cells of a brain and administering the therapeutically effective amount of the compound reduces neuron hyperexcitability in a brain of the subject.

18. The method of claim 16, wherein the compound is retigabine, a derivative thereof, or a pharmaceutically acceptable salt thereof, and the therapeutically effective amount of the compound is administered at between 0.3 mg/kg-3.0 mg/kg of body weight of the subject.

19. The method of claim 16, wherein administering the therapeutically effective amount of the compound to the subject prevents the occurrence of traumatic brain injury-induced hypersomnia in the subject.

20. A method of reducing hyperexcitability in a brain of a subject, comprising administering a therapeutically effective amount of a compound comprising an M-channel opener to a subject;
wherein the subject has experienced at least one traumatic brain injury event and the M-channel opener upregulates at least a KCNQ2 subunit, a KCNQ3 subunit, or both of an M-channel of a brain of the subject.
